(19) United States
(12) Patent Application Publication
(10) Pub. No.: US 2011/0190215 A1
(43) Pub. Date: Aug. 4, 2011

(54) THERAPEUTIC USE OF PEPTIDES

(75) Inventor: Marian L. Kruzel, Pearland, TX (US)

(73) Assignee: REGEN THERAPEUTICS PLC, London (GB)

(21) Appl. No.: 12/992,649

(22) PCT Filed: May 15, 2009
(86) PCT No.: PCT/GB2009/001251

§ 371 Date: Apr. 26, 2011

Related U.S. Application Data
(60) Provisional application No. 61/053,388, filed on May 15, 2008.
(60) Provisional application No. 61/053,394, filed on May 15, 2008.
(60) Provisional application No. 61/053,400, filed on May 15, 2008.
(60) Provisional application No. 61/053,431, filed on May 15, 2008.
(60) Provisional application No. 61/053,437, filed on May 15, 2008.

(51) Int. Cl. A61K 38/17; C07K 14/47; A61P 25/00

(52) U.S. Cl. 514/17.7; 530/326; 514/21.3; 514/21.4

(57) ABSTRACT

The amino acid sequences of peptides are disclosed. These peptides, and combinations thereof, are useful, inter alia, for the treatment of obesity, type II diabetes mellitus, hypertension, central nervous system disorders, dementia, Alzheimer's disease, asthma, and cancer,

The present invention relates to peptides and their use in therapy.

In a first embodiment, the present invention relates to peptides and their use in treating chronic central nervous system disorders, and related diseases.

Dementia is a brain disorder involving a decline in a person's cognitive functions, such as attention, language and memory, and seriously affects the person's ability to carry out daily activities. Alzheimer's disease (AD) is one of the most common forms of dementia, which involves the frontal lobe of brain. AD pathology is characterised by the neuritic plaques, microscopic foci of extracellular amyloid-beta (Aβ) deposition, and the neurofibrillary tangles, intracellular fibrils composed of hyperphosphorylated Tau protein. These Aβ deposits and Tau protein fibrils have been shown to be products of misfolded proteins associated with the increased production of reactive oxygen species (ROS) in brain. Although there has been progress in studies of the pathogenesis of AD dementia, successful preventive and therapeutic measures have yet to be developed.

There are currently several hypotheses regarding the mechanism of the development of AD. According to one hypothesis, AD is a consequence of reduced biosynthesis of the neurotransmitter acetylcholine. Previous therapies have been directed to treat the acetylcholine deficiency; however, these acetylcholine-based therapies have served to only treat symptoms of the disease and have neither halted nor reversed the progression of AD.

Recent research is based on the effects of the misfolding and aggregation of the tau protein and the Aβ peptide. These are competing hypotheses, whereby one states that the tau protein abnormalities initiate the disease cascade, while the other states that Aβ deposits cause AD. The hypothesis that the tau protein is the causative agent in AD is supported by the observation that amyloid plaque deposition does not appear to correlate well with neuron loss.

Most current research centres on Aβ as the putative causative agent of AD. Mature aggregated amyloid fibrils are highly cytotoxic, and are considered responsible for disrupting the cell's calcium ion homeostasis and inducing apoptosis, although there is some evidence that the cytotoxic species may be the intermediate, oligomeric misfolded form of Aβ, and not the soluble Aβ monomer or the mature aggregated polymer. Further studies have identified ApoE4 as a major genetic risk factor for AD; ApoE4 mediates the excess amyloid accumulation in the brain before AD symptoms arise. Thus, Aβ deposition precedes AD. Research on transgenic mice further supports Aβ as the causative agent of AD, as transgenic mice solely expressing a mutant human APP gene develop first diffuse and then fibrillar amyloid plaques, and display neuronal and microglial damage.

Thus current research into AD therapy mostly focuses on the inhibition of fibrillization, and the prevention of oligomeric assembly, and the inhibition of Amyloid Precursor Protein (APP) processing to Aβ.

There are a number of therapeutic avenues for alleviating the effects of AD dementia or delaying its progression; however, the treatments appear to only treat the symptoms. There is currently no cure to reverse AD pathology. Cholinesterase inhibitor drugs (donepezil, galantamine, tacrine, metrifonate and rivastigmine), which are intended to increase acetylcholine availability in central synapses, have been available for the treatment of AD dementia; however, these drugs only offer short term benefits during the early onset of AD symptoms. Moreover, acetylcholinesterase inhibitors have a number of disadvantages, including side effects such as nausea, anorexia, vomiting, and diarrhea.

Other approaches in treating progression of AD dementia have targeted the inflammation surrounding Aβ plaques. In this respect, use of non-steroidal anti-inflammatory drugs has been attempted in a number of preventive protocols. However, non-steroidal anti-inflammatory drugs only show some neuroprotective effect, and the overall benefit in AD treatment is not clear. There is some evidence that Ginkgo biloba extract improves cognitive functions in AD patients, although some side effects, including coma, bleeding, and seizures, have been associated with Gingko therapy. Other approaches include the development of vaccines against Aβ; vaccination of transgenic mice expressing human Aβ, a constituent of senile plaques in AD, led to clearance of Aβ from the brain. However, in human trials, some subjects developed autoimmune encephalitis, that prompted termination of the trial.

Thus there is a need for new and effective treatments for preventing and/or treating AD.

The present invention provides a use of, and a method of treatment involving, (i) a peptide comprising the amino-terminal amino acid sequence RPKHPIKHQGLPQEVLNENLLRF (SEQ ID NO 1), (i.e. Arg-Pro-Lys-His-Pro-Ile-Lys-His-Gln-Gly-Leu-Pro-Gln-Glu-Val-Leu-Asn-Glu-Asn-Leu-Leu-Arg-Phe), or a salt thereof, or (ii) a peptide analogue of (i) in which one or more amino acids have been replaced, altered and/or deleted without substantially altering the biological properties of (i), or a salt thereof, which have been found to have applications in the prevention and treatment of central nervous system disorders such as dementia and Alzheimer's disease.

These peptides may be provided in substantially isolated and/or purified form from a natural source. Alternatively, the peptides may be provided in synthetic form.

The invention further includes use of any peptide which includes the specified amino acid sequence. The invention further comprises use of any peptide which includes an amino-terminal amino acid sequence corresponding to the specified sequence. Thus, the invention encompasses use of any peptide having the N-terminal amino acid sequence RPKHPIKHQGLPQEVLNENLLRF (SEQ ID NO 1).

The peptide consisting of the sequence RPKHPIKHQGLPQEVLNENLLRF (SEQ ID NO 1) for use with the invention is present as a fragment in the protein alpha S1 kappa casein (positions 1 to 26 thereof). This peptide is commonly referred to as Isracidin.

For the avoidance of doubt, it is stated that the amino-terminal end is on the left hand side of the sequence, in accordance with the usual convention. Alternatively, the sequence may be annotated as NH₂-RPKHPIKHQGLPQEVLNENLLRF-COOH. It will be appreciated that the specified amino acid sequence may be provided with an inert amino acid sequence on the amino-terminal and/or the carboxy-terminal end thereof. The inert amino acid sequence may be a single amino acid, or a peptide containing between 2 and 5 amino acids, or a peptide containing 2 to 10 amino acids. It will be appreciated by a person skilled in the art that these inert sequences do not substantially contribute to or change the biological properties of the specified amino acid sequence, i.e. RPKHPIKHQGLPQEVLNENLLRF (SEQ ID NO 1). Furthermore, it will be appreciated by a person skilled in the art that the inert amino acid sequences may be varied. Furthermore it will be appreciated that certain inert sequences may be unsuitable. For instance, if a single alanine residue is provided at one terminal end of the specified amino acid sequence, then the skilled person will recognise that the provision of a glycine residue at the other terminal end of the peptide will be unsuitable.

The present invention is also directed to use of peptides that are polymorphs, homologues (preferably mammalian) and physiologically acceptable active derivatives of the peptide of SEQ ID NO 1, including salts thereof, which have substantially the same biological properties of the peptide of SEQ ID NO 1. These polymorphs, homologues and physiologically acceptable active derivatives may bind to antibodies (either monoclonal or polyclonal) raised against a peptide comprising or consisting of the amino acid sequence of SEQ ID NO 1, and conservatively modified peptide analogues thereof, or may have substantial sequence identity (i.e. at least about 60%) to a peptide consisting of the amino acid sequence SEQ ID NO 1 and conservatively modified peptide analogues thereof.

The term percent sequence identity refers to two or more sequences that are the same or have a specified percentage of amino acid residues that are the same, when aligned for maximum correspondence over a comparison window, in accordance with techniques well known to a person skilled in the art. For example, an amino acid sequence identity of 60% refers to sequences that have at least about 60% amino acid identity when aligned for maximum correspondence over a comparison window in accordance with techniques known to a person skilled in the art. Preferably the sequence identity is about 60%, more preferably 60-70%, more preferably 70-80%, more preferably 80-90%, more preferably about or greater than 90%.

In accordance with techniques well known to the person skilled in the art, it will be recognized that amino acid positions that are not identical may differ by conservative amino acid substitutions, where amino acids residues are substituted for other amino acid residues with similar chemical properties (e.g. size, charge and/or hydrophobicity). Conservative amino acid substitutions generally do not greatly affect the biological properties of the peptide. Examples of conservative amino acid substitutions include substitution of leucine with isoleucine, and substitution of serine with threonine. Examples of non-conservative substitutions include substitution of aspartic acid with lysine, and substitution of glycine with tryptophan.

Where sequences differ in conservative amino acid substitutions, the sequence identity may be corrected to take account for the conservative nature of the amino acid substitution. Means for making this adjustment are well known to those of skill in the art. For instance, a conservative substitution would be scored as a partial rather than a full mismatch, and thus a conservative substitution would increase the percentage sequence identity compared to a non-conservative substitution. Thus, for example, when comparing two amino acid sequences, where an identical amino acid is given a score of 1 and a non-conservative substitution is given a score of zero, a conservative substitution may be given a score between zero and 1. Techniques of scoring conservative substitutions for the purposes of determining percentage sequence identity are well known to the person skilled in the art.

The peptides may be obtained by a number of techniques. In one embodiment, they are prepared by a conventional technique for peptide synthesis, such as by solid-phase or liquid-phase peptide synthesis. Alternatively, the gene sequences encoding the peptides can be constructed by known techniques, inserted into expression vectors or plasmids, and transfected into suitable microorganisms that will express the DNA translated sequences as the peptides, whereby the peptides can be later extracted from the medium in which the microorganisms are grown.

The peptides for use in accordance with the present invention have a number of therapeutic uses. In particular the peptides have been found to be useful in the treatment of central nervous system disorders, such as dementia and Alzheimer's disease.

Thus, in accordance with the present invention there is provided a use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for the treatment of chronic disorders of the central nervous system.

In another embodiment, there is provided a use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for the prevention and treatment of dementia.

In another embodiment, there is provided a use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for the prevention and treatment of Alzheimer's disease.

In particular, it has been found that the peptides for use in accordance with the invention upregulates the gene expression of bleomycin hydrolase.

Bleomycin hydrolase (BH) is a cysteine protease. BH is involved in the processing of amyloid beta-peptides including Aβ(1-40), Aβ(1-42) and pAβ (3-42). BH cleaves Aβ(1-42) between the fourteenth histidine [His(14)] and the fifteenth glutamine [Gln(15)], and between the nineteenth and twentieth phenylalanine [Phe(19) and Phe(20)] of the protein sequence. The resulting peptides are further degraded to short intermediates by its aminopeptidase and carboxypeptidase activity. Full-length Aβs were cleaved at the C-terminal end. Bleomycin hydrolase cleaved pAβ (3-42) only between His(14) and Gln(15) by endopeptidase activity, and further processed the intermediates by carboxypeptidase activity. Fibrillar Aβ(1-40) and Aβ(1-42) were shown to be more resistant to BH than non-fibrillar peptides. Thus, advantageously, the upregulation of BH may enhance degradation of Aβ present within cells, prevent the formation of Aβ deposits within cells, and/or may lead to clearance of the Aβ deposits within the cells.

Thus the present invention provides the use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for upregulating gene expression of bleomycin hydrolase in a cell, which has been found to have applications in the prevention and treatment of central nervous system disorders such as dementia and Alzheimer's disease.

Bleomycin refers to a family of glycosylated peptide antibiotics, used as a chemotherapeutic agent in the treatment of cancers such as, inter alia, Hodgkin lymphoma and squamous cell carcinomas. Bleomycin exerts its chemotherapeutic effect by inducing DNA strand breaks, and may also inhibit thymidine incorporation into DNA. Bleomycin is toxic, thus the use of high concentrations of bleomycin during chemotherapy is limited by its side effects, including alopecia, hyperpigmentation, pulmonary fibrosis, impaired lung function, Raynaud's phenomenon, hearing loss, ototoxicity, fever and rash. Nevertheless, use of high concentrations of bleomycin to maximise the effectiveness of the chemotherapy would be desirable, especially in case of solid tumors, known to resist bleomycin therapy, partially due to overexpression of bleomycin hydrolase. One method by which this would be possible is to inject high concentrations of bleomycin locally near or in the site of the tumour and/or cancer, whilst increasing systemic amounts of bleomycin hydrolase, induced by a peptide comprising the amino-terminal amino acid sequence RPKHPIKHQGLPQEVLNENLLRF (SEQ ID NO 1), at the same time, or before, or during the course of chemotherapy. It will be possible in this way to achieve high concentrations of bleomycin near or at the site of treatment, whilst reducing the systemic toxic effects. Thus, advantageously, upregulation of bleomycin hydrolase may enhance the degradation of bleomycin in a patient. Alternatively, upregulation of bleomycin hydrolase may reduce the toxicity and side effects of a therapy comprising administration of bleomycin. Alternatively, upregulation of bleomycin hydrolase may allow for an administration of a high concentration of bleomycin local to a site of treatment.

Thus the present invention provides the use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for treating cancer. The present invention also provides the use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for adjuvant chemotherapy.

Furthermore, it has been found that the peptides for use in accordance with the invention down-regulates the gene expression of ApoE4.

APOE4 is a major genetic risk factor for AD. APOE4 appears to directly mediate the accumulation of intracellular Aβ. Recent studies have shown that Aβ production and cellular uptake appear to be modulated by apolipoprotein E (APOE) receptors and members of the low-density lipoprotein receptor (LDLR) family. Aβ undergoes rapid endocytosis upon binding to APOE, thus facilitating Aβ cellular uptake. Indeed, overexpression of an LRP minigene results in increased membrane-generated and intracellular-generated Aβ(1-42), whereas in an APOE knockout PDAPP mice overexpressing human APP V717F, (under the control of the PDGF-β promoter, which leads to the age-related brain Aβ pathology) intracellular Aβ is dramatically reduced. Thus, it appears that a large portion of the accumulated intracellular Aβ can be attributed to the interaction between Aβ, APOE and LRP. Thus, advantageously, the downregulation of ApoE4 may lower the genetic risk factor associated with APOE4. Alternatively, downregulation of ApoE4 may reduce Aβ production and/or Aβ cellular uptake. Alternatively, downregulation of ApoE4 may prevent, treat or reduce Aβ cellular accumulation.

Thus the present invention provides the use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for downregulating gene expression of ApoE4 in a cell, which has been found to have applications in the prevention and treatment of central nervous system disorders such as dementia and Alzheimer's disease.

In addition to the foregoing, there may be provided a use of the peptides to prevent and/or treat disorders or diseases associated with abnormal protein folding into amyloid or amyloid-like deposits or into pathological beta-sheet-rich precursors of such deposits to be treated or prevented, such as Alzheimer's disease, FAF, Down's syndrome, other amyloidosis disorders, human prion diseases, such as kuru, Creutzfeldt-Jakob Disease (CJD), Gerstmann-Strausslet-Scheinker Syndrome (GSS), prion associated human neurodegenerative diseases as well as animal prion diseases such as scrapie, spongiform encephalopathy, transmissible mink encephalopathy and chronic wasting. Those skilled in the art will appreciate that the above list is illustrative and not exhaustive, and that the peptides of the present invention may be used to treat other disorders associated with abnormal protein folding into amyloid or amyloid-like deposits or into pathological beta-sheet-rich precursors of such deposits.

In addition to the foregoing, there may be provided a use of the peptides to prevent and/or treat neoplastic disorders associated with amyloid or amyloid-like deposits, such as prostate, colon, brain, lung and breast cancers. Other neoplastic disorders that may be treated include tumours and cancers, including physiological conditions in mammals that are typically characterized by unregulated cell growth, such as carcinoma, lymphoma, blastoma, sarcoma, leukemia, and lymphoid malignancies. More specific examples of cancers include kidney or renal cancer, rectal cancer, colorectal cancer, ovarian cancer, liver cancer, bladder cancer, cancer of the peritoneum, hepatocellular cancer, lung cancers such as small-cell lung cancer, non-small cell lung cancer, adenocarcinoma of the lung and squamous carcinoma of the lung, head and neck cancer, glioblastoma, retinoblastoma, astrocytoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, urinary tract carcinomas, thyroid carcinomas, Wilm's tumour, gastric or stomach cancer including gastrointestinal cancer, gastrointestinal stromal tumours (GIST), pancreatic cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, squamous cell cancer (e.g. epithelial squamous cell cancer), cervical cancer, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; post-transplant lymphoproliferative disorder (PTLD), as well as abnormal vascular proliferation associated with phakomatoses, salivary gland carcinoma, vulval cancer, edema (such as that associated with brain tumours), and Meigs' syndrome. Examples of tumours include all neoplastic cell growth and proliferation, whether malignant or benign, all pre-cancerous and cancerous cells and tissues, including resistant tumours that do not respond completely, or loses or shows a reduced response over the course of cancer therapy. Those skilled in the art will appreciate that the above list is illustrative and not exhaustive, and that the peptides of the present invention may be used to treat other neoplastic disorders associated with amyloid or amyloid-like deposits.

Furthermore, it has been found that the peptides for use in accordance with the invention downregulates the gene expression of glutamate receptors. Specifically, the peptides for use in accordance with the invention downregulates the gene expression of glutamate receptor, ionotropic, N-methyl D-aspartate 2A; glutamate receptor, metabotropic 7; brain glutamate decarboxylase 2; and glutamate receptor, metabotropic 8.

Glutamate is the most prominent neurotransmitter in the body, being present in over 50% of nervous tissue. The primary glutamate receptor is an ion channel, and is specifically sensitive to N-Methyl-D-Aspartate (NMDA), which causes direct activation of the central pore of the receptor, thus depolarizing the neuron. NMDA is considered excitatory, as the neuron depolarization triggers the action potential of the neuron. However, glutamate has the potential to be highly toxic. Thus the neurotransmitter glutamate is important in both plasticity and pathology of nervous tissue. Glutamate excitotoxicity has been implicated in a number of brain disorders, including epilepsy, amyotropic lateral sclerosis, Huntington's disease, Alzheimer's disease, ischemia and trauma. Evidence suggests that an increase in glutamate neurotransmission and glutamate-glutamine cycling contributes to AD pathology and contributes to the propagation of neuronal destruction. Therefore a decrease in ionotropic glutamate receptor concentrations or subunit composition could be neuroprotective against increased glutamate levels.

Thus, advantageously, the downregulation of glutamate receptors may act as a neuroprotective against increased glutamate levels.

Thus, the present invention provides use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for the treatment of disorders characterised by glutamate excitotoxicity.

Alternatively, the present invention provides use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for down-regulating gene expression of glutamate receptors in and/or on a cell. The use of the peptides in accordance with the present invention may downregulate the gene expression of glutamate receptors glutamate receptor, ionotropic, N-methyl D-aspartate 2A; glutamate receptor, metabotropic 7; brain glutamate decarboxylase 2; and glutamate receptor, metabotropic 8.

As a result of their ability to inhibit neuronal cell death associated with N-methyl-D-aspartic acid (excitotoxicity), in accordance with the present invention, there is provided use of (i) a peptide comprising the amino-terminal amino acid sequence RPKHPIKHQGLPQEVLNENLLRF (SEQ ID NO 1), or a salt thereof, or (ii) a peptide analogue of (i) in which one or more amino acids have been replaced, altered and/or deleted without substantially altering the biological properties of (i), or a salt thereof, in the manufacture of a medicament for the treatment of a disease selected from the group comprising epilepsy, amyotropic lateral sclerosis, Huntington's disease, Alzheimer's disease, ischemia, AIDS dementia complex; neuropathic pain syndromes; olivopontocerebellar atrophy; parkinsonism and Parkinson's disease; mitochondrial abnormalities and other inherited or acquired biochemical disorders; MELAS syndrome; MERRF; Leber's disease; Wernicke's encephalopathy; Rett syndrome; homocysteinuria; hyperprolinemia; nonketotic hyperglycinemia; hydroxybutyric aminoaciduria; sulfite oxide deficiency; combined systems disease; lead encephalopathy; Alzheimer's disease; hepatic encephalopathy; Tourette's syndrome; oxidative stress induced neuronal death; Down's syndrome; developmental retardation and learning impairments; closed head trauma; dopamine toxicity; drug addiction, tolerance, and dependency.

Those of skill in the art will appreciate that the lists of neurological disorders above are illustrative and not exhaustive, and that the peptides for use in accordance with the present invention can be used to treat other neurological disorders.

A chronic disorder is a disorder that has persisted, or is expected to persist, for a long time, i.e., at least 3 months and usually at least 6 months.

The peptides may be administered prophylactically in order to help to prevent the development of disorders of the central nervous system.

The peptides for use in accordance with the invention may be used to prevent amyloidosis or to promote the dissolution of beta-amyloid aggregates (plaques), and, therefore, the peptides may be used prophylactically or in the treatment of any disease which is characterised by the development of beta-amyloid aggregates.

The peptides for use in accordance with the invention may be administered in a dosage in the range 1 nM to 10 mM. A dosage unit of about 2 μM is typical. However, the optimum dosage will, of course, depend upon the condition being treated.

The peptides may be formulated for administration in any suitable form. Thus, the peptides may be in the form of a composition, especially a pharmaceutical composition, which includes the peptides in combination with a physiologically acceptable carrier. The peptides may, for example, be formulated for oral, topical, rectal or parenteral administration. More specifically, the peptides may be formulated for administration by injection, or, preferably, in a form suitable for absorption through the mucosa of the oral/nasopharyngeal cavity, the alimentary canal or any other mucosal surface. The peptides may be formulated for administration intravenously, subcutaneously or intramuscularly. The oral formulations may be provided in a form for swallowing or, preferably, in a form for dissolving in the saliva, whereby the formulation can be absorbed in the mucous membranes of the oral/nasopharyngeal cavity. The oral formulations may be in the form of a tablet (i.e. fast dissolving tablets) for oral administration, lozenges (i.e. a sweet-like tablet in a form suitable to be retained in the mouth and sucked), or adhesive gels for rubbing into the gum. The peptides may be formulated as an adhesive plaster or patch, which may be applied to the gums. The peptides may also be formulated for application to mucous-membranes of the genito-urinary organs. The topical formulations may be provided in the form of, for example, a cream or a gel. The peptides may also be formulated as a spray for application to the nasopharyngeal or bronchial mucous surface.

The peptides may be incorporated into products like milk, yogurts, milkshake, ice cream, cheese spread and various beverage products, including sport drinks.

The invention also encompasses the selective administration of the peptides, at selected times to a patient.

In some applications it may be desirable to provide a pharmaceutical composition which contains the peptides in combination with a physiologically acceptable carrier.

The invention further embraces the use of the peptides in the manufacture of a medicament for use in any of the therapeutic applications described above.

The invention further embraces the methods of treating a mammal, in particular human, in any of the therapeutic applications described above.

### EXAMPLE 1

### Production of Synthetic Peptides

The peptides are synthesized using automated synthesizer (Advanced ChemTech model ACT 396) and a polystyrene resin (Wang resin) that has the last amino acid attached to it through a linker. All the amino acids are protected at the N-terminus with the FMOC group. The coupling reagents and all amino acids were purchased from NOVABIOCHM/EMD Biosciences, Inc., San Diego, USA. The protocol for the production of the peptide involves the following steps:

1. The resin is treated with 20% piperidine for 1×5 min and then with 1×10 min.

2. 4×1 min wash with dimethylformamide

3. 5×amino acid, 5×coupling reagent (diisopropylcarbodiimide), and 5×Hobt. (Couple for 1 hour)

4. 4×1 min wash with dimethylformamide

This cycle is repeated for each amino acid. The last cycle involves three additional steps:

5. 2×10 min with piperidine to remove the final FMOC

6. 4×1 min wash with Dimethylformamide

7. 4×5 min wash with methanol

The peptide is then cleaved form the resin with trifluroacetic acid with the following scavengers: 5% H₂O, 3% ethanedithiol, 2% thioanisole, and 1% triisopropylsilane for 2-3 hours. The peptide is then precipitated in ether and washed 5 more times with ether.

The peptide is analyzed by MALD-TOF mass spectrometry and further purified on a BIO-CAD 60 HPLC from ABI.

### EXAMPLE 2

### Cells for Microarray Procedure

TR146 buccal mucosal cells, obtained from Cancer Research U.K., were propagated in Dulbecco's modified Eagle medium (DMEM) high glucose (Gibco). The culture medium supplemented with 3.7 mg/ml NaHCO₃, 10% FCS, 50 units/ml penicillin G, and 50 mg/ml streptomycin sulphate. Cells were passaged when 90% confluence was reached. The medium was discarded, and cells were washed twice with sterile DPBS (without calcium and magnesium, Cellgro) and 0.25% trypsin-EDTA solution (Gibco) was added. The flask was placed at 37° C. for 10 minutes and then detached cells were suspended in growth medium and seeded in new flasks (dishes micro-well plates).

### EXAMPLE 3

### Preparation of Cells for Microarray Procedure

For microarray analysis, cells at 75-80% confluence in T75 flasks were treated with equimolar (2.0 μM) concentrations of a peptide consisting of the sequence RPKHPIKHQGLPQEVLNENLLRF (SEQ ID NO 1). Mock-treated cells received same volume of solvent. After 6 hours the cells were washed twice with DPBS, trypsinized, suspended in 5 ml growth media and centrifuged (800 g for 10 minutes). Cells were resuspended in 5 ml DPBS and centrifuged again. Cell pellets were used for RNA isolation.

### EXAMPLE 4

### RNA isolation for Microarray Procedure

Total RNAs were isolated with an Ambion RNAqueous Kit, and cDNAs were synthesized and purified for Affymetrix GeneChip® Human Genome Focus Array analysis. Raw data were analyzed by the Affymetrix NetAFFX Analysis Center online tools. These results provided by our Affymetrix data analysis were further analyzed through the use of Ingenuity Pathway Analysis software.

The nature of change of gene expression profile in response to the peptide consisting of the sequence RPKHPIKHQGLPQEVLNENLLRF (SEQ ID NO 1) was determined. Four result files were generated: 1. Molecular networks; 2. Biological functions and diseases; 3. Metabolic-, signalling-, and other molecular pathways; 4. Network node molecule lists. Fischer's exact test was used to calculate a p-value determining the probability that each biological function and/or disease assigned to that data set is due to chance alone.

Treatment of cells with the peptide consisting of the sequence RPKHPIKHQGLPQEVLNENLLRF (SEQ ID NO 1) altered the expression of nearly 400 genes (296 down-regulated, 101 up-regulated). A number of genes of interest displayed altered expression. These include:

Bleomycin hydrolase: upregulated;

glutamate receptors
glutamate receptor, ionotropic, N-methyl D-aspartate 2A: down-regulated;
glutamate receptor, metabotropic 7: down-regulated;
glutamate receptor, metabotropic 8: down-regulated;
brain glutamate decarboxylase 2: down-regulated;

Apolipoprotein E (APOE) and receptors: down-regulated.

Disease network analysis of gene expression profile was then carried out. Taking into consideration the nature of changes (up- and down-regulated genes), and based on the effect of each gene product, as well as their position in a specific network, the peptide was identified as having application in the prevention and/or treatment of several diseases.

The peptide of sequence RPKHPIKHQGLPQEVLNENLLRF (SEQ ID NO 1) up-regulates bleomycin hydrolase gene expression, and thus it may have significance in prevention and treatment in Alzheimer's disease. The peptide down-regulates glutamate receptor expression, thus it could have therapeutic significance in Alzheimer's disease and other central nervous system disorders (e.g., epilepsy, amyotropic lateral sclerosis, Huntington's disease, ischemia and trauma). The peptide also down-regulates Apolipoprotein E, a major genetic risk factor for Alzheimer's disease.

It will be appreciated that the invention described herein may be modified, within the scope of the claims.

In another embodiment, the present invention relates to peptides and their use in treating obesity and related disorders.

More than 65 percent of adults in the United States are overweight or obese. Obesity puts people at increased risk for chronic diseases, such as cardiovascular diseases, endocrine disorders, type II diabetes, high blood pressure, psychological disorders, respiratory disorders, stroke, infertility, osteoarthritis and several forms of cancer. Recent studies have associated a systemic inflammatory response characterized by endothelial cell dysfunction, oxidative stress, and circulating immune cell activation with obesity. For instance, adipocytes release a variety of cytokines, such as IL-1 and TNF-alpha, and cytokine-like substances, such as leptin and resistin, which appear to mediate this inflammatory response. The inflammatory response may be exacerbated by the insulin resistance that is often associated with obesity. Further research has shown that obesity may aggravate microvascular dysfunction associated with pathological states, such as sepsis.

Thus there is a need for new and effective treatments for preventing and/or treating obesity.

The present invention provides a peptide comprising the amino acid sequence FVAPFPEVFGKEKV (SEQ ID NO 2) (i.e. Phe-Val-Ala-Pro-Phe-Pro-Glu-Val-Phe-Gly-Lys-Glu-Lys-Val), or a salt thereof. Alternatively the present invention provides a peptide substantially consisting of the amino aid sequences FVAPFPEVFGKEKV (SEQ ID NO 2), or a salt thereof. Alternatively the present invention provides a peptide analogue of a peptide comprising the amino acid sequence FVAPFPEVFGKEKV (SEQ ID NO 2) in which one or more amino acids have been replaced, altered and/or deleted without substantially altering the biological properties of a peptide comprising the sequence FVAPFPEVFGKEKV (SEQ ID NO 2), or a salt thereof. These peptides have been found to be useful in the prevention and treatment of obesity, inflammatory diseases, type II diabetes, and associated diseases.

The amino acid sequence FVAPFPEVFGKEKV (SEQ ID NO 2) is homologous with the amino acid sequence, positions 39 to 52, of casein alpha S-1.

The peptides may be provided in substantially isolated and/or purified form from a natural source. Alternatively, they may be formed by a synthetic process.

For the avoidance of doubt, it is stated that the amino-terminal end is on the left hand side of the sequence, in accordance with the usual convention. Alternatively, the sequence may be annotated as NH₂-FVAPFPEVFGKEKV-COOH. It will be appreciated that the specified amino acid sequence may be provided with an inert amino acid sequence on the amino-terminal and/or the carboxy-terminal end thereof. The inert amino acid sequence may be a single amino acid, or a peptide containing between 2 and 5 amino acids, or a peptide containing 2 to 10 amino acids. It will be appreciated by a person skilled in the art that these inert sequences do not substantially contribute to or change the biological properties of the specified amino acid sequence, i.e. FVAPFPEVFGKEKV (SEQ ID NO 2). Furthermore, it will be appreciated by a person skilled in the art that the inert amino acid sequences may be varied. Furthermore it will be appreciated that certain inert sequences may be unsuitable. For instance, if a single alanine residue is provided at one terminal end of the specified amino acid sequence, then the skilled person will recognise that the provision of a glycine residue at the other terminal end of the peptide will be unsuitable.

The present invention is also directed to use of peptides that are polymorphs, homologues (preferably mammalian) and physiologically acceptable active derivatives of the peptide of SEQ ID NO 2, including salts thereof, which have substantially the same biological properties of the peptide of SEQ ID NO 2. These polymorphs, homologues and physiologically acceptable active derivatives may bind to antibodies (either monoclonal or polyclonal) raised against a peptide comprising or consisting of the amino acid sequence of SEQ ID NO 2, and conservatively modified peptide analogues thereof, or may have substantial sequence identity (i.e. at least about 60%) to a peptide consisting of the amino acid sequence SEQ ID NO 2 and conservatively modified peptide analogues thereof.

The term percent sequence identity refers to two or more sequences that are the same or have a specified percentage of amino acid residues that are the same, when aligned for maximum correspondence over a comparison window, in accordance with techniques well known to a person skilled in the art. For example, an amino acid sequence identity of 60% refers to sequences that have at least about 60% amino acid identity when aligned for maximum correspondence over a comparison window in accordance with techniques known to a person skilled in the art. Preferably the sequence identity is about 60%, more preferably 60-70%, more preferably 70-80%, more preferably 80-90%, more preferably about or greater than 90%.

In accordance with techniques well known to the person skilled in the art, it will be recognized that amino acid positions that are not identical may differ by conservative amino acid substitutions, where amino acids residues are substituted for other amino acid residues with similar chemical properties (e.g. size, charge and/or hydrophobicity). Conservative amino acid substitutions generally do not greatly affect the biological properties of the peptide. Examples of conservative amino acid substitutions include substitution of leucine with isoleucine, and substitution of serine with threonine. Examples of non-conservative substitutions include substitution of aspartic acid with lysine, and substitution of glycine with tryptophan.

Where sequences differ in conservative amino acid substitutions, the sequence identity may be corrected to take account for the conservative nature of the amino acid substitution. Means for making this adjustment are well known to those of skilled in the art. For instance, a conservative substitution would be scored as a partial rather than a full mismatch, and thus a conservative substitution would increase the percentage sequence identity compared to a non-conservative substitution. Thus, for example, when comparing two amino acid sequences, where an identical amino acid is given a score of 1 and a non-conservative substitution is given a score of zero, a conservative substitution may be given a score between zero and 1. Techniques of scoring conservative substitutions for the purposes of determining percentage sequence identity are well known to the person skilled in the art.

The peptides may be obtained by a number of techniques. In one embodiment, it is prepared by a conventional technique for peptide synthesis, such as by solid-phase or liquid-phase peptide synthesis. Alternatively, the gene sequence encoding the peptide can be constructed by known techniques, inserted into expression vectors or plasmids, and transfected into suitable microorganisms that will express the DNA translated sequences as the peptide, whereby the peptide can be later extracted from the medium in which the microorganisms are grown.

The peptides according to the present invention have a number of therapeutic uses. In particular it has been found that the peptides according to the present invention down-regulates the gene expression of resistin. Thus, in accordance with the present invention, there is provided the use of the peptides according to the invention to downregulate the gene expression of resistin in a cell.

Resistin, also known as Serine/Cysteine-rich Adipocyte-Specific Secretory Factor, is a hormone secreted by adipose tissue. When first discovered, the term resistin was coined due to the observed resistance to insulin in mice injected with this hormone. Resistin comprises a dimer of two 92 amino acid polypeptides; the pre-peptide form of resistin in human is 108 amino acids in length, with a molecular weight of about 12.5 kiloDaltons. It is one of a variety of hormones synthesized and released from adipose tissue, including adiponectin, angiotensin, estradiol, IL-6, leptin, PAI-1 and TNF-α. Resistin is thought to serve endocrine functions likely involved in insulin resistance. Further research has suggested a role for resistin to other physiological systems, for instance, obesity and energy homeostasis.

Resistin has also been implicated in the induction of inflammation. Leukocyte recruitment (for instance neutrophils and mast cells) at sites of infection or irritation due to the innate immune response results in leukocyte accumulation and secretion of inflammatory agents such as histamine, prostaglandin and pro-inflammatory cytokines; resistin has been shown to be associated in these inflammatory responses. For instance, resistin has been shown to increase gene expression of several pro-inflammatory cytokines, such as interleukin-1 (IL-1), interleukin-6 (IL-6), interleukin-12 (IL-12), and tumour necrosis factor-α (TNF-α) in an NFκB-mediated fashion. Furthermore studies have shown that resistin upregulates expression of intracellular adhesion molecule-1 (ICAM1), vascular cell-adhesion molecule-1 (VCAM1) and CCL2, all of which are involved in leukocyte recruitment to sites of infection or irritation. As resistin has been implicated in insulin resistance, resistin may well be involved in the well-known association between inflammation and insulin resistance.

Recent studies have shown a strong relationship between obesity, insulin resistance, and chronic inflammation, thereby suggesting that resistin (and/or its associated signalling pathways), at least in part, may serve as a link between obesity and type II diabetes mellitus, in addition to contributing to the inflammatory response. Nevertheless, resistin certainly bears features of a pro-inflammatory cytokine, thus resistin may well have a role in inflammatory diseases regardless of resistin's putative role in insulin resistance.

The strong relationship observed between serum resistin levels and obesity has suggested a role for resistin in energy metabolism and type II diabetes mellitus; for instance, increased serum resistin levels have been observed with increased adiposity. Other studies have demonstrated positive correlations between resistin levels and insulin resistance, as well as direct correlations between resistin levels and subjects with type II diabetes mellitus. Thus some researchers have suggested the link between increased resistin serum levels and the insulin resistance apparently associated with increased adiposity.

Thus, the downregulation of resistin may reduce the innate immune response of a cell to an infection or irritation. Alternatively, the downregulation of resistin may downregulate the expression of pro-inflammatory cytokines such as IL-1, IL-6, IL-12 and TNF-α, and downregulate the expression of ICAM1, VCAM1 and CCL2. Thus, advantageously, the peptides in accordance with the present invention may be used to prevent and/or treat inflammatory disorders.

Furthermore, the downregulation of resistin may prevent and/or treat type II diabetes mellitus. Thus, advantageously, the peptides in accordance with the present invention may be used to prevent and/or treat type II diabetes mellitus.

Furthermore, the downregulation of resistin may prevent and/or treat obesity. Thus, advantageously, the peptides in accordance with the present invention may be used to prevent and/or treat obesity.

In addition to the foregoing, the peptides of the present invention, or salts thereof, may be used to prevent and/or treat other inflammatory disorders. An inflammatory disorder may be either an acute or chronic inflammatory disorder, which can result from infections or non-infectious causes. Infectious conditions include meningitis, encephalitis, uveitis, colitis, dermatitis, and adult respiratory distress syndrome. Non-infectious causes include trauma (bumps, cuts, contusions, crush injuries), autoimmune diseases, and organ rejection episodes. Thus an inflammatory disorder may be a condition selected from a group comprising: atherosclerosis (arteriosclerosis); autoimmune conditions, such as multiple sclerosis, systemic lupus erythematosus, polymyalgia rheumatica (PMR), rheumatoid arthritis and other forms of inflammatory arthritis, Sjogren's Syndrome, progressive systemic sclerosis (scleroderma), ankylosing spondylitis, polymyositis, dermatomyositis, pemphigus, pemphigoid, Type I diabetes mellitus, myasthenia gravis, Hashimoto's thyroditis, Graves' disease, Goodpasture's disease, mixed connective tissue disease, sclerosing cholangitis, inflammatory bowel disease including Crohn's Disease (regional enteritis) and ulcerative colitis, pernicious anemia, inflammatory dermatoses; usual interstitial pneumonitis (UIP), asbestosis, silicosis, berylliosis, talcosis, all forms of pneumoconiosis, sarcoidosis (in the lung and in any other organ), desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa); inflammatory dermatoses not presumed to be autoimmune; chronic active hepatitis; delayed-type hypersensitivity reactions (e.g., poison ivy dermatitis); pneumonia or other respiratory tract inflammation due to any cause; Adult Respiratory Distress Syndrome (ARDS) from any etiology; encephalitis, with inflammatory edema; immediate hypersensitivity reactions including, but not limited to, asthma, hayfever, cutaneous allergies, acute anaphylaxis; diseases involving acute deposition of immune complexes, including, but not limited to, rheumatic fever, acute and/or chronic glomerulonephritis due to any etiology, including specifically post-infectious (e.g., post-Streptococcal) glomerulonephritis, acute exacerbations of Systemic Lupus Erythematosus; pyelonephritis; cellulitis; cystitis; acute cholecystitis; and conditions producing transient ischemia anywhere along the gastrointestinal tract, bladder, heart, or other organ, especially those prone to rupture; sequalae of organ transplantation or tissue allograft, including allograft rejection in the acute time period following allogeneic organ or tissue transplantation and chronic host-versus-graft rejection.

In addition to the foregoing, the peptides of the present invention, or salts thereof, may be used to prevent and/or treat obesity-related and obesity-associated disorders and disorders related to type II diabetes mellitus such as hyperlipidemia; dyslipidemia; abdominal obesity; hypercholesterolemia; hypertrigyceridemia; atherosclerosis; coronary heart disease; stroke; hypertension; peripheral vascular disease; vascular restenosis; nephropathy; neuropathy; inflammatory conditions, such as, but not limited to, irritable bowel syndrome, inflammatory bowel disease, including Crohn's disease and ulcerative colitis; other inflammatory conditions; pancreatitis; neurodegenerative disease; retinopathy; neoplastic conditions, such as, but not limited to adipose cell tumors, adipose cell carcinomas, such as liposarcoma; cancers, including gastric and bladder cancers; angiogenesis; Alzheimer's disease; psoriasis; and other disorders where insulin resistance is a component. The peptides of the invention may also be useful in the treatment, control and/or prevention of overeating; bulimia; elevated plasma insulin concentrations; insulin resistance; glucose tolerance; Metabolic Syndrome; lipid disorders; low HDL levels; diabetes while mitigating cardiac hypertrophy, including left ventricular hypertrophy; high LDL levels; hyperglycemia; neoplastic conditions, such as endometrial, breast, prostate, kidney and colon cancer; osteoarthritis; obstructive sleep apnea; gallstones; abnormal heart rhythms; heart arrythmias; myocardial infarction; congestive heart failure; sudden death; ovarian hyperandrogenism, (polycystic ovary disease); craniopharyngioma; the Prader-Willi Syndrome; Frohlich's syndrome; GH-deficient subjects; normal variant short stature; Turner's syndrome; and other pathological conditions showing reduced metabolic activity or a decrease in resting energy expenditure as a percentage of total fat-free mass, e.g., children with acute lymphoblastic leukemia.

Furthermore, it has been found that the peptides according to the present invention downregulate gene expression of renin and the gene network associated with renin. Thus, in accordance with the present invention, there is provided the use of the peptides according to the invention, or salts thereof, to downregulate the gene expression of renin in a cell.

Renin (angiotensinogenase) is a 340 amino acid circulating enzyme that cleaves angiotensinogen to form angiotensin I, thereby activating the renin-angiotensin system. It is released in response to low blood volume or decreased serum NaCl concentration mainly by juxtaglomerular cells in the juxtaglomerular apparatus of the kidneys, mediated via prostaglandins. Sympathetic activation of cell membrane β1- and α1-adrenergic receptors also causes renin release, most likely by altering tubular sodium content or macula densa function. Over-activation of the renin-angiotensin system leads to vasoconstriction and retention of sodium and water, thereby leading to hypertension.

Thus, the downregulation of renin may reduce the activation of the renin-angiotensin system, by reducing the rate by which angiotensin I is produced from angiotensinogen. Consequently the downregulation of the gene expression of renin may prevent and/or treat hypertension. Thus, advantageously, the peptides in accordance with the present invention may be used to prevent and/or treat hypertension.

In addition to the foregoing, the peptides of the present invention may be used to prevent and/or treat diseases associated with a dysregulation of the renin-angiotensin system, in particular diseases such as or related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, stroke, myocardial infarction, glaucoma, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases related to the renin-angiotensin system.

Recent studies have demonstrated that several physiological and pathological brain functions are mediated by the brain's own renin-angiotensin system. In particular, research has focussed on the neurobiological links between the renin-angiotensin system and Alzheimer's disease with a view to investigating the pathogenesis of the disease. Analyses of longitudinal and cross-sectional studies suggest a correlation between high blood pressure and dementia; thus the use of anti-hypertensives has been proposed to reduce incidence of dementia. Thus, although most approaches in the therapeutic strategy against Alzheimer's disease concentrate on decreasing Aβ load, the renin-angiotensin system is also of recognized importance in the pathogenesis of Alzheimer's disease.

Thus, the downregulation of renin may reduce the activation of the renin-angiotensin system, by reducing the rate by which angiotensin I is produced from angiotensinogen. Consequently the downregulation of the gene expression of renin and the reduction in hypertension may prevent and/or treat Alzheimer's disease. Thus, advantageously, the peptides in accordance with the present invention, or salts thereof, may be used to prevent and/or treat Alzheimer's disease.

A chronic disorder is a disorder that has persisted, or is expected to persist, for a long time, i.e., at least 3 months and usually at least 6 months.

The peptides also have diagnostic and research applications. For example, a synthetic peptide of SEQ ID NO 2, as well as the corresponding antibodies described below, may be used to recognise pathological processes occurring in a host. These processes may be induced by excessive production or inhibition of the peptide or the antibodies. Once the pathological process associated with a particular level of the peptide or the antibodies is known, measuring the production of the peptide and the antibodies in body fluids may be used to determine pathological processes taking place in the host. This may occur, for example, in lactating mothers during various infections or drug treatments.

According to another aspect of the invention, we provide the use of the peptides as a dietary supplement. This dietary supplement may be particularly useful for obese patients, patients with type II diabetes mellitus, patients with hypertension or patients with Alzheimer's disease. In an aspect of the invention, we provide a dietary supplement comprising an orally ingestible blend of the peptides in combination with a physiologically acceptable carrier. The dietary supplement may be provided in liquid or solid form; the dietary supplement may suitably be provided in the form of a tablet. The dietary supplement may be provided in the form of a baby food formula. The dietary supplement may include, as an additive, lactoferrin and/or selenium and/or a group of cytokines containing members of the interferon family.

The peptides of the invention may be administered prophylactically in order to help to prevent the development of obesity, inflammation disorders, hypertension, and/or other related diseases.

The peptides in accordance with the invention may be administered in a dosage in the range 1 nM to 10 mM. A dosage unit of about 2 μM is typical. However, the optimum dosage will, of course, depend upon the condition being treated.

The peptides in accordance with the invention may be formulated for administration in any suitable form. Thus, the use in accordance with the invention may be in the form of a composition, especially a pharmaceutical composition, which includes the peptide in combination with a physiologically acceptable carrier. The peptide may, for example, be formulated for oral, topical, rectal or parenteral administration. More specifically, the peptide may be formulated for administration by injection, or, preferably, in a form suitable for absorption through the mucosa of the oral/nasopharyngeal cavity, the alimentary canal or any other mucosal surface. The peptide may be formulated for administration intravenously, subcutaneously, or intramuscularly. The oral formulations may be provided in a form for swallowing or, preferably, in a form for dissolving in the saliva, whereby the formulation can be absorbed in the mucous membranes of the oral/nasopharyngeal cavity. The oral formulations may be in the form of a tablet (i.e. fast dissolving tablets) for oral administration, lozenges (i.e. a sweet-like tablet in a form suitable to be retained in the mouth and sucked), or adhesive gels for rubbing into the gum. The peptide may be formulated as an adhesive plaster or patch, which may be applied to the gums. The peptide may also be formulated for application to mucous-membranes of the genito-urinary organs. The topical formulations may be provided in the form of, for example, a cream or a gel. The peptide may also be formulated as a spray for application to the nasopharyngeal or bronchial mucous surface.

The peptides may be incorporated into products like milk, yogurts, milkshake, ice cream, cheese spread and various beverage products, including sport drinks.

In another aspect, the invention provides an antibody for the peptides, and provides compositions containing said antibodies. In particular the invention provides the antibodies in substantially isolated form. The antibodies can be produced by injecting a suitable subject, such as a rabbit, with the peptides (with a suitable adjuvant), then recovering the antibodies from the subject after allowing time for them to be produced. It is possible to test that the correct antibody has been produced by ELISA (enzyme-linked immunosorbent assay) using the synthetic peptide as antigens. The antibodies have potential uses in therapy, as a diagnostic tool and as a research tool. The antibodies can be produced in accordance with the methods described in example 3 of WO00/75173.

The invention also encompasses the selective administration of the peptides, at selected times to a patient.

In some applications it may be desirable to provide a pharmaceutical composition which contains the peptides in combination with a physiologically acceptable carrier.

The invention further embraces the use of the peptides in the manufacture of a medicament for use in any of the therapeutic applications described above.

The invention further embraces the methods of treating a mammal, in particular human, in any of the therapeutic applications described above.

### EXAMPLE 1

### Production of Synthetic Peptides

The peptides are synthesized using automated synthesizer (Advanced ChemTech model ACT 396) and a polystyrene resin (Wang resin) that has the last amino acid attached to it through a linker. All the amino acids are protected at the N-terminus with the FMOC group. The coupling reagents and all amino acids were purchased from NOVABIOCHM/EMD Biosciences, Inc., San Diego, USA. The protocol for the production of the peptide involves the following steps:

1. The resin is treated with 20% piperidine for 1×5 min and then with 1×10 min.

2. 4×1 min wash with dimethylformamide

3. 5×amino acid, 5×coupling reagent (diisopropylcarbodiimide), and 5×Hobt. (Couple for 1 hour)

4. 4×1 min wash with dimethylformamide

This cycle is repeated for each amino acid. The last cycle involves three additional steps:

5. 2×10 min with piperidine to remove the final FMOC

6. 4×1 min wash with Dimethylformamide

7. 4×5 min wash with methanol

The peptide is then cleaved form the resin with trifluroacetic acid with the following scavengers: 5% H2O, 3% ethanedithiol, 2% thioanisole, and 1% triisopropylsilane for 2-3 hours. The peptide is then precipitated in ether and washed 5 more times with ether.

The peptide is analyzed by MALD-TOF mass spectrometry and further purified on a BIO-CAD 60 HPLC from ABI.

### EXAMPLE 2

### Cells for Microarray Procedure

TR146 buccal mucosal cells, obtained from Cancer Research U.K., were propagated in Dulbecco's modified Eagle medium (DMEM) high glucose (Gibco). The culture medium supplemented with 3.7 mg/ml NaHCO₃, 10% FCS, 50 units/ml penicillin G, and 50 mg/ml streptomycin sulphate. Cells were passaged when 90% confluence was reached. The medium was discarded, and cells were washed twice with sterile DPBS (without calcium and magnesium, Cellgro) and 0.25% trypsin-EDTA solution (Gibco) was added. The flask was placed at 37° C. for 10 minutes and then detached cells were suspended in growth medium and seeded in new flasks (dishes micro-well plates).

### EXAMPLE 3

### Preparation of Cells for Microarray Procedure

For microarray analysis, cells at 75-80% confluence in T75 flasks were treated with equimolar (2.0 μM) concentrations of peptides. Mock-treated cells received same volume of solvent. After 6 hours the cells were washed twice with DPBS, trypsinized, suspended in 5 ml growth media and centrifuged (800 g for 10 minutes). Cells were resuspended in 5 ml DPBS and centrifuged again. Cell pellets were used for RNA isolation.

### EXAMPLE 4

### RNA isolation for Microarray Procedure

Total RNAs were isolated with an Ambion RNAqueous Kit, and cDNAs were synthesized and purified for Affymetrix GeneChip® Human Genome Focus Array analysis. Raw data were analyzed by the Affymetrix NetAFFX Analysis Center online tools. These results provided by our Affymetrix data analysis were further analyzed through the use of Ingenuity Pathway Analysis software.

The nature of change of gene expression profile in response to the peptide was determined. Four result files were generated: 1. Molecular networks; 2. Biological functions and diseases; 3. Metabolic-, signalling-, and other molecular pathways; 4. Network node molecule lists. Fischer's exact test was used to calculate a p-value determining the probability that each biological function and/or disease assigned to that data set is due to chance alone.

A number of genes of interest displayed altered expression. These include:

Resistin: down-regulated;

Renin (angiotensinogenase): down-regulated.

Disease network analysis of gene expression profile was then carried out. Taking into consideration the nature of changes (up- and down-regulated genes), and based on the effect of each gene product, as well as their position in a specific network, the peptide was identified as having application in the prevention and/or treatment of several diseases.

The peptide downregulates resistin gene expression, and thus it may have significance in prevention and treatment in disorders such as obesity, inflammatory disorders, type II diabetes mellitus, and disorders related thereto. The peptide also down-regulates renin expression, thus it may have therapeutic significance in the treatment of hypertension and disorders related thereto.

It will be appreciated that the invention described herein may be modified, within the scope of the claims.

In another embodiment, the present invention relates to peptides and their use in treating disorders of the immune system and related disorders.

Asthma is a condition of the lungs, or the bronchi, in which the airways occasionally constrict, become inflamed, and/or produce large amounts of mucus. Asthmatics may display symptoms such as wheezing, shortness of breath, chest tightness, and coughing. Some individuals may exhibit chronic asthma, whilst others may exhibit intermittent episodes of asthma in response to stimuli. Asthma episodes may be triggered by a variety of stimuli, including exposure to allergens, medications, air pollution (such as smoke, vehicle exhaust and industrial chemicals), hormonal changes, exercise, respiratory infections, or changes in humidity and/or temperature of the air. Severe acute asthmatic episodes (also known as asthma attacks), if untreated, may lead to respiratory arrest, or even death.

Asthma is rapidly becoming a major problem due to its increasing prevalence; about 3.6% of children under 18 years of age were diagnosed with asthma in 1980 in the US, and the prevalence rose to 9% in 2001; about 2% of the Swiss suffered from asthma around 1975 to 1980, whereas the prevalence increased to about 8% in 2005. In the UK, there is an asthmatic in one in five households in the UK. Asthma is not restricted to individuals of the developed world; there is estimated to be between 15 and 20 million asthmatics in India alone.

A number of studies have focussed on the causes of asthma. Many have linked asthma and air quality; in particular, ozone pollution. Other studies have linked the lack of exposure of individuals to pathogens to an increased risk of asthma.

Asthma is treatable, although there is currently no cure. Treatments include limiting exposure to or desensitizing a patient's response to stimuli, or administration of drugs prior to or during an asthma episode.

There is a need for new and effective treatments for preventing and/or treating asthma.

The present invention provides a peptide comprising the amino acid sequence EPVLGPVRGPFPI (SEQ ID NO 5) (i.e. Glu-Pro-Val-Leu-Gly-Pro-Val-Arg-Gly-Pro-Phe-Pro-Ile, or a salt thereof. Alternatively the present invention provides a peptide substantially consisting of the amino aid sequences EPVLGPVRGPFPI (SEQ ID NO 5), or a salt thereof. Alternatively the present invention provides a peptide analogue of a peptide comprising the amino acid sequence EPVLGPVRGPFPI (SEQ ID NO 5) in which one or more amino acids have been replaced, altered and/or deleted without substantially altering the biological properties of a peptide comprising the sequence EPVLGPVRGPFPI (SEQ ID NO 5), or a salt thereof. These peptides have been found to be useful in the prevention and/or treatment of inflammatory and immunological diseases, in particular, asthma.

The amino acid sequence EPVLGPVRGPFPI (SEQ ID NO 5) is homologous with the amino acid sequence, positions 195 to 207, of casein beta.

The peptides may be provided in substantially isolated and/or purified form from a natural source. Alternatively, they may be formed by a synthetic process.

For the avoidance of doubt, it is stated that the amino-terminal end is on the left hand side of the sequence, in accordance with the usual convention. Alternatively, the sequence may be annotated as NH₂-EPVLGPVRGPFPI-COOH. It will be appreciated that the specified amino acid sequence may be provided with an inert amino acid sequence on the amino-terminal and/or the carboxy-terminal end thereof. The inert amino acid sequence may be a single amino acid, or a peptide containing between 2 and 5 amino acids, or a peptide containing 2 to 10 amino acids. It will be appreciated by a person skilled in the art that these inert sequences do not substantially contribute to or change the biological properties of the specified amino acid sequence, i.e. EPVLGPVRGPFPI (SEQ ID NO 5). Furthermore, it will be appreciated by a person skilled in the art that the inert amino acid sequences may be varied. Furthermore it will be appreciated that certain inert sequences may be unsuitable. For instance, if a single alanine residue is provided at one terminal end of the specified amino acid sequence, then the skilled person will recognise that the provision of a glycine residue at the other terminal end of the peptide will be unsuitable.

The present invention is also directed to use of peptides that are polymorphs, homologues (preferably mammalian) and physiologically acceptable active derivatives of the peptide of SEQ ID NO 5, including salts thereof, which have substantially the same biological properties of the peptide of SEQ ID NO 5. These polymorphs, homologues and physiologically acceptable active derivatives may bind to antibodies (either monoclonal or polyclonal) raised against a peptide comprising or consisting of the amino acid sequence of SEQ ID NO 5, and conservatively modified peptide analogues thereof, or may have substantial sequence identity (i.e. at least about 60%) to a peptide consisting of the amino acid sequence SEQ ID NO 5 and conservatively modified peptide analogues thereof.

The term percent sequence identity refers to two or more sequences that are the same or have a specified percentage of amino acid residues that are the same, when aligned for maximum correspondence over a comparison window, in accordance with techniques well known to a person skilled in the art. For example, an amino acid sequence identity of 60% refers to sequences that have at least about 60% amino acid identity when aligned for maximum correspondence over a comparison window in accordance with techniques known to a person skilled in the art. Preferably the sequence identity is about 60%, more preferably 60-70%, more preferably 70-80%, more preferably 80-90%, more preferably about or greater than 90%.

In accordance with techniques well known to the person skilled in the art, it will be recognized that amino acid positions that are not identical may differ by conservative amino acid substitutions, where amino acids residues are substituted for other amino acid residues with similar chemical properties (e.g. size, charge and/or hydrophobicity). Conservative amino acid substitutions generally do not greatly affect the biological properties of the peptide. Examples of conservative amino acid substitutions include substitution of leucine with isoleucine, and substitution of serine with threonine. Examples of non-conservative substitutions include substitution of aspartic acid with lysine, and substitution of glycine with tryptophan.

Where sequences differ in conservative amino acid substitutions, the sequence identity may be corrected to take account for the conservative nature of the amino acid substitution. Means for making this adjustment are well known to those of skilled in the art. For instance, a conservative substitution would be scored as a partial rather than a full mismatch, and thus a conservative substitution would increase the percentage sequence identity compared to a non-conservative substitution. Thus, for example, when comparing two amino acid sequences, where an identical amino acid is given a score of 1 and a non-conservative substitution is given a score of zero, a conservative substitution may be given a score between zero and 1. Techniques of scoring conservative substitutions for the purposes of determining percentage sequence identity are well known to the person skilled in the art.

The peptides may be obtained by a number of techniques. In one embodiment, it is prepared by a conventional technique for peptide synthesis, such as by solid-phase or liquid-phase peptide synthesis. Alternatively, the gene sequence encoding the peptide can be constructed by known techniques, inserted into expression vectors or plasmids, and transfected into suitable microorganisms that will express the DNA translated sequences as the peptide, whereby the peptide can be later extracted from the medium in which the microorganisms are grown.

The peptides according to the present invention have a number of therapeutic uses. In particular it has been found that the peptides according to the present invention downregulate the gene expression of GABA (gamma-aminobutyric acid) receptors, including rho 2; gamma-aminobutyric acid A receptor (GABAAR), beta 2 gamma-aminobutyric acid receptor, theta; and glutamate decarboxylase 2 (pancreatic islets, lung and brain, 65 kDa [kilo Daltons]). Thus, in accordance with the present invention, there is provided the use of the peptides according to the invention to downregulates the gene expression of various GABA (gamma-aminobutyric acid) receptors, including rho 2; gamma-aminobutyric acid A receptor (GABAAR), beta 2 gamma-aminobutyric acid receptor, theta; and glutamate decarboxylase 2 (pancreatic islets, lung and brain, 65 kDa), in a cell.

GABA (gamma-aminobutyric acid) is a prominent inhibitory neurotransmitter, for which there are three main classes of receptor: GABA_{A}, GABA_{B} ionotropic and GABA_{C} metabotropic receptors. Pulmonary epithelial cells express both GABA_{A} receptors and the GABA synthetic enzyme glutamic acid decarboxylase; studies have shown that there is an excitatory, as opposed to an inhibitory, GABAergic system in airway epithelial cells.

An asthmatic displays a number of symptoms during an attack, including airway goblet cell hyperplasia and excessive production of mucus. The upregulation of the expression of GABAergic signalling molecules in response to an allergen challenge has been observed in animal and human asthma models. Intranasal administration of selective GABA_{A} receptor inhibitors suppressed goblet cell hyperplasia and mucus overproduction.

Thus, downregulation of the gene expression of various GABA receptors [rho 2; GABA A receptor, beta 2 GABA receptor, theta; and glutamate decarboxylase 2 (pancreatic islets, lung and brain, 65 kDa)] may prevent and/or reduce goblet cell hyperplasia and/or mucus overproduction. Alternatively, downregulation of the gene expression of various GABA receptors may downregulate the expression of GABAergic signalling molecules in response to an allergen challenge in a cell. Alternatively, downregulation of the gene expression of various GABA receptors may prevent and/or treat asthma. Alternatively, downregulation of on the gene expression of various GABA receptors may prevent and/or treat an inflammatory and/or an immunological disorder. Thus, advantageously, the peptides in accordance with the present invention may be used to prevent and/or treat asthma, including allergen-induced asthma, viral-induced asthma, cold-induced asthma, pollution-induced asthma and exercise-induced asthma.

In addition to the foregoing, the peptides of the present invention, or salts thereof, may be used to prevent and/or treat other pulmonary disorders, including bronchospasm; chronic obstructive pulmonary disease, including chronic bronchitis with normal airflow; rhinitis, including allergic rhinitis; and for inducing bronchodilation. Other diseases may be treated with the peptides of the present invention, or salts thereof, including disorders characterized by acute pulmonary vasoconstriction, for instance when resulting from pneumonia, traumatic injury, aspiration or inhalation injury; adult respiratory distress syndrome; post-cardiac surgery; acute pulmonary edema; acidosis inflammation of the lung; persistent pulmonary hypertension in newborn; hyaline membrane disease; fat embolism in the lung; heparin-protamine reactions; perinatal aspiration syndrome; acute mountain sickness; sepsis; acute pulmonary hypertension; acute pulmonary thromboembolism; status asthmaticus or hypoxia, including iatrogenic hypoxia; and other forms of reversible pulmonary vasoconstriction. Other diseases may be treated with the peptides of the present invention, or salts thereof, including disorders characterized by inflammation of the lung, including those associated with the migration into the lung of non-resident cell types; cystic fibrosis; pigeon fancier's disease; allergic eye diseases, including allergic conjunctivitis, vernal conjunctivitis, vernal keratoconjunctivitis, and giant papillary conjunctivitis; emphysema; bullous disease; asthmatic bronchitis; farmer's lung; chronic bronchitis with airway obstruction, or chronic obstructive bronchitis; and other diseases which are characterized by inflammation of the lung and/or excess mucus secretion.

In addition to the foregoing, the peptides of the present invention, or salts thereof, may be used to prevent and/or treat other inflammatory disorders. An inflammatory disorder may be either an acute or chronic inflammatory disorder, which can result from infections or non-infectious causes. Infectious conditions include meningitis, encephalitis, uveitis, colitis, dermatitis, and adult respiratory distress syndrome. Non-infectious causes include trauma (burns, cuts, contusions, crush injuries), autoimmune diseases, and organ rejection episodes. Thus an inflammatory disorder may be a condition selected from a group comprising: atherosclerosis (arteriosclerosis); autoimmune conditions, such as multiple sclerosis, systemic lupus erythematosus, polymyalgia rheumatica (PMR), rheumatoid arthritis and other forms of inflammatory arthritis, Sjogren's Syndrome, progressive systemic sclerosis (scleroderma), ankylosing spondylitis, polymyositis, dermatomyositis, pemphigus, pemphigoid, Type I diabetes mellitus, myasthenia gravis, Hashimoto's thyroiditis, Graves' disease, Goodpasture's disease, mixed connective tissue disease, sclerosing cholangitis, inflammatory bowel disease including Crohn's Disease (regional enteritis) and ulcerative colitis, pernicious anemia, inflammatory dermatoses; usual interstitial pneumonitis (UIP), asbestosis, silicosis, berylliosis, talcosis, all forms of pneumoconiosis, sarcoidosis (in the lung and in any other organ), desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa); inflammatory dermatoses not presumed to be autoimmune; chronic active hepatitis; delayed-type hypersensitivity reactions (e.g., poison ivy dermatitis); pneumonia or other respiratory tract inflammation due to any cause; Adult Respiratory Distress Syndrome (ARDS) from any etiology; encephalitis, with inflammatory edema; immediate hypersensitivity reactions including, but not limited to hayfever, cutaneous allergies, acute anaphylaxis; diseases involving acute deposition of immune complexes, including, but not limited to, rheumatic fever, acute and/or chronic glomerulonephritis due to any etiology, including specifically post-infectious (e.g., post-Streptococcal) glomerulonephritis, acute exacerbations of Systemic Lupus Erythematosus; pyelonephritis; cellulitis; cystitis; acute cholecystitis; and conditions producing transient ischemia anywhere along the gastrointestinal tract, bladder, heart, or other organ, especially those prone to rupture; sequalae of organ transplantation or tissue allograft, including allograft rejection in the acute time period following allogeneic organ or tissue transplantation and chronic host-versus-graft rejection.

A chronic disorder is a disorder that has persisted, or is expected to persist, for a long time, i.e., at least 3 months and usually at least 6 months.

The peptides also have diagnostic and research applications. For example, a synthetic peptide of SEQ ID NO 5, as well as the corresponding antibodies described below, may be used to recognise pathological processes occurring in a host. These processes may be induced by excessive production or inhibition of the peptide or the antibodies. Once the pathological process associated with a particular level of the peptide or the antibodies is known, measuring the production of the peptide and the antibodies in body fluids may be used to determine pathological processes taking place in the host. This may occur, for example, in lactating mothers during various infections or drug treatments.

According to another aspect of the invention, we provide the use of the peptides as a dietary supplement. This dietary supplement may be particularly useful for patients with inflammatory diseases, immunological diseases, asthma, pulmonary diseases, and other disorders related thereto. In an aspect of the invention, we provide a dietary supplement comprising an orally ingestible blend of the peptides in combination with a physiologically acceptable carrier. The dietary supplement may be provided in liquid or solid form; the dietary supplement may suitably be provided in the form of a tablet. The dietary supplement may be provided in the form of a baby food formula. The dietary supplement may include, as an additive, lactoferrin and/or selenium and/or a group of cytokines containing members of the interferon family.

The peptides of the invention may be administered prophylactically in order to help to prevent the development of inflammatory diseases, immunological diseases, asthma, pulmonary diseases, and other disorders related thereto.

The peptides in accordance with the invention may be administered in a dosage in the range 1 nM to 10 mM. A dosage unit of about 2 μM is typical. However, the optimum dosage will, of course, depend upon the condition being treated.

The peptides in accordance with the invention may be formulated for administration in any suitable form. Thus, the use in accordance with the invention may be in the form of a composition, especially a pharmaceutical composition, which includes the peptide in combination with a physiologically acceptable carrier. The peptide may, for example, be formulated for oral, topical, rectal or parenteral administration. More specifically, the peptide may be formulated for administration by injection, or, preferably, in a form suitable for absorption through the mucosa of the oral/nasopharyngeal cavity, the alimentary canal or any other mucosal surface. The peptide may be formulated for administration intravenously, subcutaneously, or intramuscularly. The oral formulations may be provided in a form for swallowing or, preferably, in a form for dissolving in the saliva, whereby the formulation can be absorbed in the mucous membranes of the oral/nasopharyngeal cavity. The oral formulations may be in the form of a tablet (i.e. fast dissolving tablets) for oral administration, lozenges (i.e. a sweet-like tablet in a form suitable to be retained in the mouth and sucked), or adhesive gels for rubbing into the gum. The peptide may be formulated as an adhesive plaster or patch, which may be applied to the gums. The peptide may also be formulated for application to mucous-membranes of the genito-urinary organs. The topical formulations may be provided in the form of, for example, a cream or a gel. The peptide may also be formulated as a spray for application to the nasopharyngeal or bronchial mucous surface.

The peptides may be incorporated into products like milk, yogurts, milkshake, ice cream, cheese spread and various beverage products, including sport drinks.

In another aspect, the invention provides an antibody for the peptides, and provides compositions containing said antibodies. In particular the invention provides the antibodies in substantially isolated form. The antibodies can be produced by injecting a suitable subject, such as a rabbit, with the peptides (with a suitable adjuvant), then recovering the antibodies from the subject after allowing time for them to be produced. It is possible to test that the correct antibody has been produced by ELISA (enzyme-linked immunosorbent assay) using the synthetic peptide as antigens. The antibodies have potential uses in therapy, as a diagnostic tool and as a research tool. The antibodies can be produced in accordance with the methods described in example 3 of WO00/75173.

The invention also encompasses the selective administration of the peptides, at selected times to a patient.

In some applications it may be desirable to provide a pharmaceutical composition which contains the peptides in combination with a physiologically acceptable carrier.

The invention further embraces the use of the peptides in the manufacture of a medicament for use in any of the therapeutic applications described above.

The invention further embraces the methods of treating a mammal, in particular human, in any of the therapeutic applications described above.

### EXAMPLE 1

### Production of Synthetic Peptides

The peptides are synthesized using automated synthesizer (Advanced ChemTech model ACT 396) and a polystyrene resin (Wang resin) that has the last amino acid attached to it through a linker. All the amino acids are protected at the N-terminus with the FMOC group. The coupling reagents and all amino acids were purchased from NOVABIOCHM/EMD Biosciences, Inc., San Diego, USA. The protocol for the production of the peptide involves the following steps:

1. The resin is treated with 20% piperidine for 1×5 min and then with 1×10 min.

2. 4×1 min wash with dimethylformamide

3. 5×amino acid, 5×coupling reagent (diisopropylcarbodiimide), and 5×Hobt. (Couple for 1 hour)

4. 4×1 min wash with dimethylformamide

This cycle is repeated for each amino acid. The last cycle involves three additional steps:

5. 2×10 min with piperidine to remove the final FMOC

6. 4×1 min wash with Dimethylformamide

7. 4×5 min wash with methanol

The peptide is then cleaved form the resin with trifluroacetic acid with the following scavengers: 5% H2O, 3% ethanedithiol, 2% thioanisole, and 1% triisopropylsilane for 2-3 hours. The peptide is then precipitated in ether and washed 5 more times with ether.

The peptide is analyzed by MALD-TOF mass spectrometry and further purified on a BIO-CAD 60 HPLC from ABI.

### EXAMPLE 2

### Cells for Microarray Procedure

TR146 buccal mucosal cells, obtained from Cancer Research U.K., were propagated in Dulbecco's modified Eagle medium (DMEM) high glucose (Gibco). The culture medium supplemented with 3.7 mg/ml NaHCO₃, 10% FCS, 50 units/ml penicillin G, and 50 mg/ml streptomycin sulphate. Cells were passaged when 90% confluence was reached. The medium was discarded, and cells were washed twice with sterile DPBS (without calcium and magnesium, Cellgro) and 0.25% trypsin-EDTA solution (Gibco) was added. The flask was placed at 37° C. for 10 minutes and then detached cells were suspended in growth medium and seeded in new flasks (dishes micro-well plates).

### EXAMPLE 3

### Preparation of Cells for Microarray Procedure

For microarray analysis, cells at 75-80% confluence in T75 flasks were treated with equimolar (2.0 μM) concentrations of peptide of SEQ ID NO 5. Mock-treated cells received same volume of solvent. After 6 hours the cells were washed twice with DPBS, trypsinized, suspended in 5 ml growth media and centrifuged (800 g for 10 minutes). Cells were resuspended in 5 ml DPBS and centrifuged again. Cell pellets were used for RNA isolation.

### EXAMPLE 4

### RNA isolation for Microarray Procedure Total RNAs were isolated with an Ambion RNAqueous Kit, and cDNAs were synthesized and purified for Affymetrix GeneChip® Human Genome Focus Array analysis. Raw data were analyzed by the Affymetrix NetAFFX Analysis Center online tools. These results provided by our Affymetrix data analysis were further analyzed through the use of Ingenuity Pathway Analysis software.

The nature of change of gene expression profile in response to the peptide was determined. Four result files were generated: 1. Molecular networks; 2. Biological functions and diseases; 3. Metabolic-, signalling-, and other molecular pathways; 4. Network node molecule lists. Fischer's exact test was used to calculate a p-value determining the probability that each biological function and/or disease assigned to that data set is due to chance alone.

A number of genes of interest displayed altered expression. In particular, the gene expression of the following genes was downregulated following treatment with the peptide of SEQ ID NO 5:

rho 2;

gamma-aminobutyric acid A receptor (GABAAR)

beta 2 gamma-aminobutyric acid receptor, theta

glutamate decarboxylase 2 (pancreatic islets, lung and brain, 65 kDa)

Disease network analysis of gene expression profile was then carried out. Taking into consideration the nature of changes (up- and down-regulated genes), and based on the effect of each gene product, as well as their position in a specific network, the peptide of SEQ ID NO 5 was identified as having application in the prevention and/or treatment of several diseases.

The peptide of SEQ ID NO 5 downregulated the gene expression of rho 2, gamma-aminobutyric acid A receptor (GABAAR), beta 2 gamma-aminobutyric acid receptor, theta, and glutamate decarboxylase 2 (pancreatic islets, lung and brain, 65 kDa), and thus it may have significance in prevention and treatment in disorders such as inflammatory disorders, immunological disorders, asthma, pulmonary disorders, and other disorders related thereto.

It will be appreciated that the invention described herein may be modified, within the scope of the claims.

In another embodiment, the present invention relates to peptides and their use in treating disorders of the central nervous system, cancer and related disorders.

Dementia is a brain disorder involving a decline in a person's cognitive functions, such as attention, language and memory, and seriously affects the person's ability to carry out daily activities. Alzheimer's disease (AD) is one of the most common forms of dementia, which involves the frontal lobe of brain. AD pathology is characterised by the neuritic plaques, microscopic foci of extracellular amyloid-beta (Aβ) deposition, and the neurofibrillary tangles, intracellular fibrils composed of hyperphosphorylated Tau protein. These Aβ deposits and Tau protein fibrils have been shown to be products of misfolded proteins associated with the increased production of reactive oxygen species (ROS) in brain. Although there has been progress in studies of the pathogenesis of AD dementia, successful preventive and therapeutic measures have yet to be developed.

There are currently several hypotheses regarding the mechanism of the development of AD. According to one hypothesis, AD is a consequence of reduced biosynthesis of the neurotransmitter acetylcholine. Previous therapies have been directed to treat the acetylcholine deficiency; however, these acetylcholine-based therapies have served to only treat symptoms of the disease and have neither halted nor reversed the progression of AD.

Recent research is based on the effects of the misfolding and aggregation of the tau protein and the Aβ peptide. These are competing hypotheses, whereby one states that the tau protein in abnormalities initiate the disease cascade, while the other states that Aβ deposits cause AD. The hypothesis that the tau protein is the causative agent in AD is supported by the observation that amyloid plaque deposition does not appear to correlate well with neuron loss.

Most current research centres on Aβ as the putative causative agent of AD. Mature aggregated amyloid fibrils are highly cytotoxic, and are considered responsible for disrupting the cell's calcium ion homeostasis and inducing apoptosis, although there is some evidence that the cytotoxic species may be the intermediate, oligomeric misfolded form of Aβ, and not the soluble Aβ monomer or the mature aggregated polymer. Further studies have identified ApoE4 is a major genetic risk factor for AD; ApoE4 mediates the excess amyloid accumulation in the brain before AD symptoms arise. Thus, Aβ deposition precedes AD. Research on transgenic mice further supports Aβ as the causative agent of AD, as transgenic mice solely expressing a mutant human APP gene develop first diffuse and then fibrillar amyloid plaques, and display neuronal and microglial damage.

Thus current research into AD therapy mostly focuses on the inhibition of fibrillization, and the prevention of oligomeric assembly, and the inhibition of Amyloid Precursor Protein (APP) processing to Aβ.

There are a number of therapeutic avenues for alleviating the effects of AD dementia or delaying its progression; however, the treatments appear to only treat the symptoms. There is currently no cure to reverse AD pathology. Cholinesterase inhibitor drugs (donepezil, galantamine, tacrine, metrifonate and rivastigmine), which are intended to increase acetylcholine availability in central synapses, have been available for the treatment of AD dementia; however, these drugs only offer short term benefits during the early onset of AD symptoms. Moreover, acetylcholinesterase inhibitors have a number of disadvantages, including side effects such as nausea, anorexia, vomiting, and diarrhea.

Other approaches in treating progression of AD dementia have targeted the inflammation surrounding Aβ plaques. In this respect, use of non-steroidal anti-inflammatory drugs has been attempted in a number of preventive protocols. However, non-steroidal anti-inflammatory drugs only show some neuroprotective effect, and the overall benefit in AD treatment is not clear. There is some evidence that Ginkgo biloba extract improves cognitive functions in AD patients, although some side effects, including coma, bleeding, and seizures, have been associated with Gingko therapy. Other approaches include the development of vaccines against Aβ; vaccination of transgenic mice expressing human Aβ, a constituent of senile plaques in AD, led to clearance of Aβ from the brain. However, in human trials, some subjects developed autoimmune encephalitis, that prompted termination of the trial.

Thus there is a need for new and effective treatments for preventing and/or treating AD.

The present invention provides a peptide comprising the amino acid sequence RMPLPPRGCPAAAPWS (SEQ ID NO 10) (i.e. Arg-Met-Pro-Leu-Pro-Pro-Arg-Gly-Cys-Pro-Ala-Ala-Ala-Pro-Trp-Ser), or a salt thereof. Alternatively the present invention provides a peptide substantially consisting of the amino aid sequences RMPLPPRGCPAAAPWS (SEQ ID NO 10), or a salt thereof. Alternatively the present invention provides a peptide analogue of a peptide comprising the amino acid sequence RMPLPPRGCPAAAPWS (SEQ ID NO 10) in which one or more amino acids have been replaced, altered and/or deleted without substantially altering the biological properties of a peptide comprising the sequence RMPLPPRGCPAAAPWS (SEQ ID NO 10), or a salt thereof. These peptides have been found to be useful in the prevention and treatment of central nervous system disorders such as dementia and Alzheimer's disease, and in the prevention and treatment of cancer.

The peptides according to the present invention have a number of therapeutic uses. In particular it has been found that the peptides according to the present invention downregulate the gene expression of Amyloid Beta (A4) Precursor Protein. Thus, in accordance with the present invention, there are provided peptides for downregulating the gene expression of Amyloid Beta (A4) Precursor Protein in a cell.

Amyloid Beta (A4) Precursor Protein (APP), also known as peptidase nexin-II, is expressed in many tissues, and is found at high concentrations in the synapses of neurons. Whilst its function is not known, it has been implicated in synapse formation and repair, as well as neural plasticity. Several isoforms, ranging in amino acid length from 365 to 770, have been identified in humans. Proteins with homology to APP have been identified in all mammals, as well as Drosophila and C. Elegans. APP is an integral membrane protein, comprising two extracellular structural domains. The membrane-spanning domain of the protein is not particularly well conserved between species, and studies have not been able to attribute this membrane-spanning domain with APP function.

APP may be cleaved by any one of alpha, beta and gamma secretase. When cleaved by both gamma and beta secretase, APP produces a short 39-42 amino acid peptide called amyloid beta (Aβ). The abnormally folded amyloid fibrillar form of Aβ is the primary component of amyloid senile plaques found in the brain cells of patient suffering from Alzheimer's disease. Mutations in critical regions of APP, including the region that generates amyloid beta, are known to cause familial susceptibility to Alzheimer's disease. For example, several mutations outside the Aβ region associated with familial Alzheimer's have been found to dramatically increase production of Aβ. Gamma secretase has also been identified as a major genetic risk factor for Alzheimer's.

The presence of APP in lipid rafts has been linked to its amyloidogenic processing. For instance, when APP molecules are located outside lipid raft regions of membranes, they are cleaved by the non-amyloidogenic alpha secretase, whereas when APP molecules occupy a lipid raft, they are more accessible to, and differentially cleaved by, beta secretase. Observations that high cholesterol is a major risk factor for Alzheimer's disease has thus been linked with cholesterol's role in lipid raft maintenance.

Thus, downregulation of APP may reduce the amount of cellular APP available for amyloidogenic processing. Alternatively, downregulation of APP may reduce the amount Aβ produced in a cell. Alternatively, downregulation of APP may reduce the amount of Aβ available for in the cell for the formation of amyloid plaques. Alternatively, downregulation of APP may inhibit the formation of amyloid plaques. Alternatively, downregulation of APP may prevent, delay and/or reverse the progression of Alzheimer's disease. Thus, advantageously, the peptides in accordance with the present invention may be used to prevent and/or treat central nervous system disorders such as dementia and Alzheimer's disease.

Furthermore, it has been found that the peptides according to the present invention downregulate gene expression of protein-tyrosine phosphatase, receptor-type, F. Thus, in accordance with the present invention, there are provided peptides, or salts thereof, for downregulating the gene expression of protein-tyrosine phosphatase, receptor-type, F, in a cell.

Protein-tyrosine phosphatases regulate phosphorylation state of proteins by enzymatically removing phosphates from phosphorylated tyrosines. Previous studies of cancers have identified 83 somatic mutations in 6 protein-tyrosine phosphatases affecting 26% of colorectal cancers and smaller fractions of lung, breast, and gastric cancers. Fifteen mutations resulted in truncated proteins lacking phosphatase activity. Overexpression of wild type protein-tyrosine phosphatases in human cancer cells inhibited cell growth, thus suggesting that mutated tyrosine phosphatases are tumorigenic.

Thus, the downregulation of protein-tyrosine phosphatase, receptor-type, F in a patient expressing mutated protein-tyrosine phosphatase, receptor-type, F and/or suffering from cancer, in particular colorectal, lung, breast and/or gastric cancer, may reduce the tumorigenic nature of mutated protein-tyrosine phosphatase, receptor-type, F. Alternatively, the downregulation of protein-tyrosine phosphatase, receptor-type, F in a patient expressing mutated protein-tyrosine phosphatase, receptor-type, F and/or suffering from cancer, in particular colorectal, lung, breast and/or gastric cancer, may reduce the cancer cell survival and/or inhibit cancer cell growth. Thus, advantageously, the peptides in accordance with the present invention may be used to prevent and/or treat cancer, in particular, colorectal, lung, breast and/or gastric cancer.

Furthermore, it has been found that the peptides according to the present invention upregulate gene expression of Stathmin-1. Thus, in accordance with the present invention, there are provided peptides, or salts thereof, for the upregulation of gene expression of Stathmin-1 in a cell.

The cytoskeleton comprises a number of components, including microtubules. Microtubules provide structural support to the cell, as well as playing a role in, inter alia, mitosis and cytokinesis. Microtubules are formed by the polymerisation of free α, β-tubulin dimers. Stathmin-1 is a 17 kiloDalton protein which controls microtubule formation by regulating the assembly and disassembly of tubulin. Stathmin-1 is able to bind with two α, β-tubulin dimers through the Stathmin-like domain (SLD) to form the T2S complex. Whilst free α, β-tubulin is able to polymerise, α, β-tubulin bound in the T2S complex is not able to polymerise. Thus, by controlling the amount of free cellular α, β-tubulin, Stathmin-1 is able to regulate microtubule assembly.

Stathmin-1 regulation is under the control of various protein kinases that respond to specific cell signals. Four serine residues serve as phosphorylation sites on Stathmin-1; phosphorylation of these residues result in weakened Stathmin-1-tubulin binding, thereby increasing the amount of free cellular tubulin available for microtubule assembly.

Mitosis is initiated by Stathmin-1 phosphorylation and the formation of the mitotic spindle, which is comprised of a bundle of microtubules. Stathmin-1 is then dephosphorylated during cytokinesis, thereby preventing the cell from reinitiating the cell cycle. Thus microtubule assembly and Stathmin-1 regulation is closely linked to cell growth and the cell cycle.

As mutated Stathmin-1 can cause uncontrolled cell proliferation, Stathmin-1 is also known as oncoprotein 18 (op18). Improper regulation of the mitotic spindle due to mutated Stathmin-1 can result uncontrolled cycling of the cell cycle, thereby leading to unregulated cell growth characteristic of cancer cells.

Thus, the upregulation of Stathmin-1 may reduce the amount of free α, β-tubulin available in the cell. Alternatively, the upregulation of Stathmin-1 may reduce the rate of microtubule assembly and/or increase the rate of microtubule disassembly. Alternatively, the upregulation of Stathmin-1 may inhibit the formation of the mitotic spindle. Alternatively, the upregulation of Stathmin-1 may inhibit the initiation of mitosis and/or the progression of the cell cycle. Alternatively, the upregulation of Stathmin-1 may inhibit unregulated cell proliferation and/or growth. Alternatively, the upregulation of Stathmin-1 may inhibit cancer cell growth. Alternatively, the upregulation of Stathmin-1 in a patient suffering from cancer may inhibit cancer cell growth and/or proliferation. Alternatively, the upregulation of Stathmin-1 in a patient suffering from a cancer characterised by oncoprotein 18 activity, may inhibit growth and/or proliferation of cancer cells characterised by oncoprotein 18 activity. Alternatively, the upregulation of Stathmin-1 in a patient suffering from cancer may prevent and/or treat cancer.

In addition to the foregoing, the peptides of the present invention may be used to prevent and/or treat tumours and cancers, including physiological conditions in mammals that are typically characterized by unregulated cell growth, such as carcinoma, lymphoma, blastoma, sarcoma, leukemia, and lymphoid malignancies. More specific examples of cancers include kidney or renal cancer, breast cancer, colon cancer, rectal cancer, colorectal cancer, ovarian cancer, prostate cancer, liver cancer, bladder cancer, cancer of the peritoneum, hepatocellular cancer, lung cancer including small-cell lung cancer, non-small cell lung cancer, adenocarcinoma of the lung and squamous carcinoma of the lung, head and neck cancer, glioblastoma, retinoblastoma, astrocytoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, urinary tract carcinomas, thyroid carcinomas, Wilm's tumour, gastric or stomach cancer including gastrointestinal cancer, gastrointestinal stromal tumours (GIST), pancreatic cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, squamous cell cancer (e.g. epithelial squamous cell cancer), cervical cancer, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; post-transplant lymphoproliferative disorder (PTLD), as well as abnormal vascular proliferation associated with phakomatoses, salivary gland carcinoma, vulval cancer, edema (such as that associated with brain tumours), and Meigs' syndrome. Examples of tumours include all neoplastic cell growth and proliferation, whether malignant or benign, all pre-cancerous and cancerous cells and tissues, including resistant tumours that do not respond completely, or loses or shows a reduced response over the course of cancer therapy. The person skilled in the art will recognise that the present invention may be used in the prevention and/or treatment of other cancers and tumours.

In an alternative embodiment, the present invention provides a combination comprising (1a) a peptide comprising the amino acid sequence RMPLPPRGCPAAAPWS (SEQ ID NO 10), or a salt thereof, or (1b) a peptide substantially consisting of the amino aid sequences RMPLPPRGCPAAAPWS (SEQ ID NO 10), or a salt thereof, or (1c) a peptide analogue of a peptide comprising the amino acid sequence RMPLPPRGCPAAAPWS (SEQ ID NO 10) in which one or more amino acids have been replaced, altered and/or deleted without substantially altering the biological properties of a peptide comprising the sequence RMPLPPRGCPAAAPWS (SEQ ID NO 10), or a salt thereof, and (2a) a peptide comprising the amino acid sequence RPKHPIKHQGLPQEVLNENLLRF (SEQ ID NO 1), (i.e. Arg-Pro-Lys-His-Pro-Ile-Lys-His-Gln-Gly-Leu-Pro-Gln-Glu-Val-Leu-Asn-Glu-Asn-Leu-Leu-Arg-Phe), or a salt thereof, or (2b) a peptide substantially consisting of the amino aid sequences RPKHPIKHQGLPQEVLNENLLRF (SEQ ID NO 1), or a salt thereof, or (2c) a peptide analogue of a peptide comprising the amino acid sequence RPKHPIKHQGLPQEVLNENLLRF (SEQ ID NO 1) in which one or more amino acids have been replaced, altered and/or deleted without substantially altering the biological properties of a peptide comprising the sequence RPKHPIKHQGLPQEVLNENLLRF (SEQ ID NO 1), or a salt thereof. The combination in accordance with the present invention has been found to have applications in the prevention and treatment of central nervous system disorders such as dementia and Alzheimer's disease, in the prevention and treatment of neoplastic disorders such as cancers, and in adjuvant chemotherapy.

The amino acid sequence RPKHPIKHQGLPQEVLNENLLRF (SEQ ID NO 1) is homologous with the amino acids of casein alpha S-1, positions 1 to 26 thereof. A peptide consisting of this amino acid sequence is commonly known as Isracidin.

In particular, it has been found that the peptide RPKHPIKHQGLPQEVLNENLLRF (SEQ ID NO 1) upregulates the gene expression of bleomycin hydrolase.

Bleomycin hydrolase (BH) is a cysteine protease. BH is involved in the processing of amyloid beta-peptides including Aβ(1-40), Aβ(1-42) and pAβ (3-42). BH cleaves Aβ(1-42) between the fourteenth histidine [His(14)] and the fifteenth glutamine [Gln(15)], and between the nineteenth and twentieth phenylalanine [Phe(19) and Phe(20)] of the protein sequence. The resulting peptides are further degraded to short intermediates by its aminopeptidase and carboxypeptidase activity. Full-length Aβs were cleaved at the C-terminal end. Bleomycin hydrolase cleaved pAβ (3-42) only between His(14) and Gln(15) by endopeptidase activity, and further processed the intermediates by carboxypeptidase activity. Fibrillar Aβ(1-40) and Aβ(1-42) were shown to be more resistant to BH than non-fibrillar peptides.

Thus, advantageously, the upregulation of BH may enhance degradation of Aβ present within cells, prevent the formation of Aβ deposits within cells, and/or may lead to clearance of the Aβ deposits within the cells.

Bleomycin refers to a family of glycosylated peptide antibiotics, which may be used as chemotherapeutic agents in the treatment of cancers such as, inter alia, Hodgkin lymphoma and squamous cell carcinomas. Bleomycin induces DNA strand breaks, and may also inhibit thymidine incorporation into DNA. Bleomycin is toxic, thus the use of high concentrations of bleomycin during chemotherapy is limited by its side effects, including alopecia, hyperpigmentation, pulmonary fibrosis, impaired lung function, Raynaud's phenomenon, hearing loss, ototoxicity, fever and rash. Nevertheless, use of high concentrations of bleomycin in chemotherapy is desirable in order to maximise the effectiveness of the treatment. This may be achieved by injecting high concentrations of bleomycin locally near or in the site of the tumour and/or cancer, whilst increasing systemic amounts of bleomycin hydrolase at the same time. In this way it may be possible to achieve high bleomycin concentrations near or at the site of treatment for effective chemotherapy, whilst reducing the systemic toxic effects of bleomycin.

Thus, advantageously, upregulation of bleomycin hydrolase may enhance the degradation of bleomycin in a patient. Alternatively, upregulation of bleomycin hydrolase may reduce the toxicity and side effects of a therapy comprising administration of bleomycin. Alternatively, upregulation of bleomycin hydrolase may allow for an administration of a high concentration of bleomycin local to a site of treatment.

Furthermore, it has been found that the peptide RPKHPIKHQGLPQEVLNENLLRF (SEQ ID NO 1) down-regulates the gene expression of ApoE4.

APOE4 is a major genetic risk factor for Alzheimer's disease (AD). APOE4 appears to directly mediate the accumulation of intracellular Aft Recent studies have shown that Aβ production and cellular uptake appear to be modulated by apolipoprotein E (APOE) receptors and members of the low-density lipoprotein receptor (LDLR) family. Aβ undergoes rapid endocytosis upon binding to APOE, thus facilitating Aβ cellular uptake. Indeed, overexpression of an LRP minigene results in increased membrane-generated and intracellular-generated Aβ(1-42), whereas in an APOE knockout PDAPP mice overexpressing human APP V717F, (under the control of the PDGF-β promoter, which leads to the age-related brain Aβ pathology) intracellular Aβ is dramatically reduced. Thus, it appears that a large portion of the accumulated intracellular Aβ can be attributed to the interaction between A13, APOE and LRP.

Thus, advantageously, the downregulation of ApoE4 may lower the genetic risk factor associated with APOE4. Alternatively, downregulation of ApoE4 may reduce Aβ production and/or Aβ cellular uptake. Alternatively, downregulation of ApoE4 may prevent, treat or reduce Aβ cellular accumulation.

Furthermore, it has been found that the peptide RPKHPIKHQGLPQEVLNENLLRF (SEQ ID NO 1) for use in accordance with the invention downregulates the gene expression of glutamate receptors. Specifically, the peptide RPKHPIKHQGLPQEVLNENLLRF (SEQ ID NO 1) downregulates the gene expression of glutamate receptor, ionotropic, N-methyl D-aspartate 2A; glutamate receptor, metabotropic 7; brain glutamate decarboxylase 2; and glutamate receptor, metabotropic 8.

Glutamate is the most prominent neurotransmitter in the body, being present in over 50% of nervous tissue. The primary glutamate receptor is an ion channel, and is specifically sensitive to N-Methyl-D-Aspartate (NMDA), which causes direct action of the central pore of the receptor, thus depolarizing the neuron. NMDA is considered excitatory, as the neuron depolarization triggers the action potential of the neuron. However, glutamate has the potential to be highly toxic. Thus the neurotransmitter glutamate is important in both plasticity and pathology of nervous tissue. Glutamate excitotoxicity has been implicated in a number of brain disorders, including epilepsy, amyotropic lateral sclerosis, Huntington's disease, Alzheimer's disease, ischemia and trauma. Evidence suggests that an increase in glutamate neurotransmission and glutamate-glutamine cycling contributes to AD pathology and contributes to the propagation of neuronal destruction. Therefore a decrease in ionotropic glutamate receptor concentrations or subunit composition could be neuroprotective against increased glutamate levels.

Thus, advantageously, the downregulation of glutamate receptors may act as a neuroprotective against increased glutamate levels.

Thus the combinations of peptides in accordance with the present invention may be useful in the treatment and/or prevention of central nervous system disorders such as dementia and Alzheimer's disease. The peptides in accordance with the present invention, when administered in combination to a patient suffering from disorders such as central nervous system disorders, dementia and/or Alzheimer's disease, may affect the body physiologically in a synergistic manner, such that the physiological effect of the combination is greater than the sum of the physiological effect of the peptides when administered alone. The synergistic effect of the combinations of the peptides of the present invention may result from the combination of effects of the peptides on different physiological targets. For instance, a combination comprising two peptides consisting of the sequences RMPLPPRGCPAAAPWS (SEQ ID NO 10) and RPKHPIKHQGLPQEVLNENLLRF (SEQ ID NO 1), when administered to a patient suffering from a central nervous system disorder such as Alzheimer's disease, would elicit a plurality of beneficial physiological effects in the patient, such as, inter alia, upregulation of gene expression of bleomycin hydrolase, downregulation of the gene expression of ApoE4, downregulation of the gene expression of APP, and downregulation of glutamate receptors. The synergy of the combined beneficial physiological response resulting from the administration of the combinations of peptides in accordance with the present invention may result in an improvement in the treatment and/or prevention of central nervous system disorders such as dementia and Alzheimer's disease, compared to administration of each peptide separately.

In addition to the foregoing, there are provided combinations of peptides in accordance with the present invention to prevent and/or treat disorders or diseases associated with abnormal protein folding into amyloid or amyloid-like deposits or into pathological beta-sheet-rich precursors of such deposits to be treated or prevented, such as Alzheimer's disease, FAF, Down's syndrome, other amyloidosis disorders, human prion diseases, such as kuru, Creutzfeldt-Jakob Disease (CJD), Gerstmann-Strausslet-Scheinker Syndrome (GSS), prion associated human neurodegenerative diseases as well as animal prion diseases such as scrapie, spongiform encephalopathy, transmissible mink encephalopathy and chronic wasting. Those skilled in the art will appreciate that the above list is illustrative and not exhaustive, and that the peptides of the present invention may be used to treat other disorders associated with abnormal protein folding into amyloid or amyloid-like deposits or into pathological beta-sheet-rich precursors of such deposits.

In addition to the foregoing, there may be provided a use of the peptides to prevent and/or treat neoplastic disorders associated with amyloid or amyloid-like deposits, such as prostate, colon, brain, lung and breast cancers. Other neoplastic disorders that may be treated include tumours and cancers, including physiological conditions in mammals that are typically characterized by unregulated cell growth, such as carcinoma, lymphoma, blastoma, sarcoma, leukemia, and lymphoid malignancies. More specific examples of cancers include kidney or renal cancer, rectal cancer, colorectal cancer, ovarian cancer, liver cancer, bladder cancer, cancer of the peritoneum, hepatocellular cancer, lung cancers such as small-cell lung cancer, non-small cell lung cancer, adenocarcinoma of the lung and squamous carcinoma of the lung, head and neck cancer, glioblastoma, retinoblastoma, astrocytoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, urinary tract carcinomas, thyroid carcinomas, Wilm's tumour, gastric or stomach cancer including gastrointestinal cancer, gastrointestinal stromal tumours (GIST), pancreatic cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, squamous cell cancer (e.g. epithelial squamous cell cancer), cervical cancer, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; post-transplant lymphoproliferative disorder (PTLD), as well as abnormal vascular proliferation associated with phakomatoses, salivary gland carcinoma, vulval cancer, edema (such as that associated with brain tumours), and Meigs' syndrome. Examples of tumours include all neoplastic cell growth and proliferation, whether malignant or benign, all pre-cancerous and cancerous cells and tissues, including resistant tumours that do not respond completely, or loses or shows a reduced response over the course of cancer therapy. Those skilled in the art will appreciate that the above list is illustrative and not exhaustive, and that the peptides of the present invention may be used to treat other neoplastic disorders associated with amyloid or amyloid-like deposits.

In addition to the foregoing, there may be provided combinations of peptides in accordance with the present invention to prevent and/or treat disorders or diseases associated with glutamate excitotoxicity, such as epilepsy, amyotropic lateral sclerosis, Huntington's disease, Alzheimer's disease, ischemia, AIDS dementia complex; neuropathic pain syndromes; olivopontocerebellar atrophy; parkinsonism and Parkinson's disease; mitochondrial abnormalities and other inherited or acquired biochemical disorders; MELAS syndrome; MERRF; Leber's disease; Wernicke's encephalopathy; Rett syndrome; homocysteinuria; hyperprolinemia; nonketotic hyperglycinemia; hydroxybutyric aminoaciduria; sulfite oxide deficiency; combined systems disease; lead encephalopathy; Alzheimer's disease; hepatic encephalopathy; Tourette's syndrome; oxidative stress induced neuronal death; Down's syndrome; developmental retardation and learning impairments; closed head trauma; dopamine toxicity; drug addiction, tolerance, and dependency.

The peptides of the present invention may be provided in substantially isolated and/or purified form from a natural source. Alternatively, they may be formed by a synthetic process.

For the avoidance of doubt, it is stated that the amino-terminal end is on the left hand side of the sequence, in accordance with the usual convention. Alternatively, the sequence may be annotated as NH₂-RMPLPPRGCPAAAPWS-COOH (SEQ ID NO 10) or NH₂-RPKHPIKHQGLPQEVLNENLLRF-COOH (SEQ ID NO 1). It will be appreciated that the specified amino acid sequences may be provided with an inert amino acid sequence on the amino-terminal and/or the carboxy-terminal end thereof. The inert amino acid sequence may be a single amino acid, or a peptide containing between 2 and 5 amino acids, or a peptide containing 2 to 10 amino acids. It will be appreciated by a person skilled in the art that these inert sequences do not substantially contribute to or change the biological properties of the specified amino acid sequence, i.e. RMPLPPRGCPAAAPWS (SEQ ID NO 10). Furthermore, it will be appreciated by a person skilled in the art that the inert amino acid sequences may be varied. Furthermore it will be appreciated that certain inert sequences may be unsuitable. For instance, if a single alanine residue is provided at one terminal end of the specified amino acid sequence, then the skilled person will recognise that the provision of a glycine residue at the other terminal end of the peptide will be unsuitable.

The present invention is also directed to peptides that are polymorphs, homologues (preferably mammalian) and physiologically acceptable active derivatives of the peptide of SEQ ID NO 10 and SEQ ID NO 1, including salts thereof, which have substantially the same biological properties of the peptide of SEQ ID NO 10 and SEQ ID NO 1, respectively. These polymorphs, homologues and physiologically acceptable active derivatives may bind to antibodies (either monoclonal or polyclonal) raised against a peptide comprising or consisting of the amino acid sequence of SEQ ID NO 10 and SEQ ID NO 1, respectively, and conservatively modified peptide analogues thereof, or may have substantial sequence identity (i.e. at least about 60%) to a peptide consisting of the amino acid sequence SEQ ID NO 10 and SEQ ID NO 1, respectively, and conservatively modified peptide analogues thereof.

The term percent sequence identity refers to two or more sequences that are the same or have a specified percentage of amino acid residues that are the same, when aligned for maximum correspondence over a comparison window, in accordance with techniques well known to a person skilled in the art. For example, an amino acid sequence identity of 60% refers to sequences that have at least about 60% amino acid identity when aligned for maximum correspondence over a comparison window in accordance with techniques known to a person skilled in the art. Preferably the sequence identity is about 60%, more preferably 60-70%, more preferably 70-80%, more preferably 80-90%, more preferably about or greater than 90%.

In accordance with techniques well known to the person skilled in the art, it will be recognized that amino acid positions that are not identical may differ by conservative amino acid substitutions, where amino acids residues are substituted for other amino acid residues with similar chemical properties (e.g. size, charge and/or hydrophobicity). Conservative amino acid substitutions generally do not greatly affect the biological properties of the peptide. Examples of conservative amino acid substitutions include substitution of leucine with isoleucine, and substitution of serine with threonine. Examples of non-conservative substitutions include substitution of aspartic acid with lysine, and substitution of glycine with tryptophan.

Where sequences differ in conservative amino acid substitutions, the sequence identity may be corrected to take account for the conservative nature of the amino acid substitution. Means for making this adjustment are well known to those of skilled in the art. For instance, a conservative substitution would be scored as a partial rather than a full mismatch, and thus a conservative substitution would increase the percentage sequence identity compared to a non-conservative substitution. Thus, for example, when comparing two amino acid sequences, where an identical amino acid is given a score of 1 and a non-conservative substitution is given a score of zero, a conservative substitution may be given a score between zero and 1. Techniques of scoring conservative substitutions for the purposes of determining percentage sequence identity are well known to the person skilled in the art.

The peptides may be obtained by a number of techniques. In one embodiment, it is prepared by a conventional technique for peptide synthesis, such as by solid-phase or liquid-phase peptide synthesis. Alternatively, the gene sequence encoding the peptide can be constructed by known techniques, inserted into expression vectors or plasmids, and transfected into suitable microorganisms that will express the DNA translated sequences as the peptide, whereby the peptide can be later extracted from the medium in which the microorganisms are grown.

A chronic disorder is a disorder that has persisted, or is expected to persist, for a long time, i.e., at least 3 months and usually at least 6 months.

The peptides also have diagnostic and research applications. For example, a synthetic peptide of SEQ ID NO 10 or SEQ ID NO 1, as well as the corresponding antibodies described below, may be used to recognise pathological processes occurring in a host. These processes may be induced by excessive production or inhibition of the peptide or the antibodies. Once the pathological process associated with a particular level of the peptide or the antibodies is known, measuring the production of the peptide and the antibodies in body fluids may be used to determine pathological processes taking place in the host. This may occur, for example, in lactating mothers during various infections or drug treatments.

According to another aspect of the invention, we provide the use of the peptides as a dietary supplement. This dietary supplement may be particularly useful for patients with central nervous system disorders, dementia, Alzheimer's disease, cancer, including colorectal, lung, breast and gastric cancers, and disorders related thereto. In an aspect of the invention, we provide a dietary supplement comprising an orally ingestible blend of the peptides in combination with a physiologically acceptable carrier. The dietary supplement may be provided in liquid or solid form; the dietary supplement may suitably be provided in the form of a tablet. The dietary supplement may be provided in the form of a baby food formula. The dietary supplement may include, as an additive, lactoferrin and/or selenium and/or a group of cytokines containing members of the interferon family.

The peptides of the invention may be administered prophylactically in order to help to prevent the development of central nervous system disorders, dementia, Alzheimer's disease, cancer, including colorectal, lung, breast and gastric cancers, and disorders related thereto.

The peptides in accordance with the invention may be administered in a dosage in the range 1 nM to 10 mM. A dosage unit of about 2 μM is typical. However, the optimum dosage will, of course, depend upon the condition being treated.

Each peptide of the combination of peptides in accordance with the present invention may be administered concomitantly or sequentially, in any therapeutically appropriate combination.

When the combination of peptides is administered concomitantly, the combination may be administered as a single pharmaceutical composition comprising the peptides, or as separate compositions administered at the same time. Each peptide is preferably present in equimolar quantities in the combination. However, it will be appreciated by the person skilled in the art that the relative amounts of the peptides in the combination may be varied. For instance, when two peptides are present in the combination, the molar ratios of each peptide may vary from 1:1000, 1:100, 1:10, 1:5, 1:4, 1:3, 1:2, 2:1, 3:1, 4:1, 5:1, 10:1, 100:1 and 1000:1. The skilled person will appreciate other ratios may be desirable, depending on the therapeutic application. When there are 3 or more peptides present in the combination, they are preferably present in equimolar amounts. Alternatively, the relative amounts of each peptide may vary, depending on the therapeutic application.

When the peptides of the combination are administered sequentially, each peptide is preferably administered within a biologically relevant time frame. Methods of sequential administration include administration of a peptide of the combination as soon as administration of another of the combination is administered; and administration of a peptide of the combination during the period when a patient is experiencing the biological effects of the administration of another peptide of the combination. Preferably equimolar amounts of each peptide are administered. However, it will be appreciated by the person skilled in the art that different molar quantities of each peptide may be administered. Different molar quantities may be administered by varying the relative frequency of administration of each peptide, or the relative amount of each peptide delivered in a single administration.

The peptides in accordance with the invention may be formulated for administration in any suitable form. Thus, the use in accordance with the invention may be in the form of a composition, especially a pharmaceutical composition, which includes the peptide in combination with a physiologically acceptable carrier. The peptide may, for example, be formulated for oral, topical, rectal or parenteral administration. More specifically, the peptide may be formulated for administration by injection, or, preferably, in a form suitable for absorption through the mucosa of the oral/nasopharyngeal cavity, the alimentary canal or any other mucosal surface. The peptide may be formulated for administration intravenously, subcutaneously, or intramuscularly. The oral formulations may be provided in a form for swallowing or, preferably, in a form for dissolving in the saliva, whereby the formulation can be absorbed in the mucous membranes of the oral/nasopharyngeal cavity. The oral formulations may be in the form of a tablet (i.e. fast dissolving tablets) for oral administration, lozenges (i.e. a sweet-like tablet in a form suitable to be retained in the mouth and sucked), or adhesive gels for rubbing into the gum. The peptide may be formulated as an adhesive plaster or patch, which may be applied to the gums. The peptide may also be formulated for application to mucous-membranes of the genito-urinary organs. The topical formulations may be provided in the form of, for example, a cream or a gel. The peptide may also be formulated as a spray for application to the nasopharyngeal or bronchial mucous surface.

The peptides may be incorporated into products like milk, yogurts, milkshake, ice cream, cheese spread and various beverage products, including sport drinks.

In another aspect, the invention provides an antibody for the peptides, and provides compositions containing said antibodies. In particular the invention provides the antibodies in substantially isolated form. The antibodies can be produced by injecting a suitable subject, such as a rabbit, with the peptides (with a suitable adjuvant), then recovering the antibodies from the subject after allowing time for them to be produced. It is possible to test that the correct antibody has been produced by ELISA (enzyme-linked immunosorbent assay) using the synthetic peptide as antigens. The antibodies have potential uses in therapy, as a diagnostic tool and as a research tool. The antibodies can be produced in accordance with the methods described in example 3 of WO00/75173.

The invention also encompasses the selective administration of the peptides, at selected times to a patient.

In some applications it may be desirable to provide a pharmaceutical composition which contains the peptides in combination with a physiologically acceptable carrier.

The invention further embraces the use of the peptides in the manufacture of a medicament for use in any of the therapeutic applications described above.

The invention further embraces the methods of treating a mammal, in particular human, in any of the therapeutic applications described above.

### EXAMPLE 1

### Production of Synthetic Peptides

The peptides are synthesized using automated synthesizer (Advanced ChemTech model ACT 396) and a polystyrene resin (Wang resin) that has the last amino acid attached to it through a linker. All the amino acids are protected at the N-terminus with the FMOC group. The coupling reagents and all amino acids were purchased from NOVABIOCHM/EMD Biosciences, Inc., San Diego, USA. The protocol for the production of the peptide involves the following steps:

1. The resin is treated with 20% piperidine for 1×5 min and then with 1×10 min.

2. 4×1 min wash with dimethylformamide

3. 5×amino acid, 5×coupling reagent (diisopropylcarbodiimide), and 5×Hobt. (Couple for 1 hour)

4. 4×1 min wash with dimethylformamide

This cycle is repeated for each amino acid. The last cycle involves three additional steps:

5. 2×10 min with piperidine to remove the final FMOC

6. 4×1 min wash with Dimethylformamide

7. 4×5 min wash with methanol

The peptide is then cleaved form the resin with trifluroacetic acid with the following scavengers: 5% H2O, 3% ethanedithiol, 2% thioanisole, and 1% triisopropylsilane for 2-3 hours. The peptide is then precipitated in ether and washed 5 more times with ether.

The peptide is analyzed by MALD-TOF mass spectrometry and further purified on a BIO-CAD 60 HPLC from ABI.

### EXAMPLE 2

### Cells for Microarray Procedure

TR146 buccal mucosal cells, obtained from Cancer Research U.K., were propagated in Dulbecco's modified Eagle medium (DMEM) high glucose (Gibco). The culture medium supplemented with 3.7 mg/ml NaHCO₃, 10% FCS, 50 units/ml penicillin G, and 50 mg/ml streptomycin sulphate. Cells were passaged when 90% confluence was reached. The medium was discarded, and cells were washed twice with sterile DPBS (without calcium and magnesium, Cellgro) and 0.25% trypsin-EDTA solution (Gibco) was added. The flask was placed at 37° C. for 10 minutes and then detached cells were suspended in growth medium and seeded in new flasks (dishes micro-well plates).

### EXAMPLE 3

### Preparation of Cells for Microarray Procedure

For microarray analysis, cells at 75-80% confluence in T75 flasks were treated with equimolar (2.0 μM) concentrations of a peptide consisting of the sequence RMPLPPRGCPAAAPWS (SEQ ID NO 10) or RPKHPIKHQGLPQEVLNENLLRF (SEQ ID NO 1). Mock-treated cells received same volume of solvent. After 6 hours the cells were washed twice with DPBS, trypsinized, suspended in 5 ml growth media and centrifuged (800 g for 10 minutes). Cells were resuspended in 5 ml DPBS and centrifuged again. Cell pellets were used for RNA isolation.

### EXAMPLE 4

### RNA isolation for Microarray Procedure

Total RNAs were isolated with an Ambion RNAqueous Kit, and cDNAs were synthesized and purified for Affymetrix GeneChip® Human Genome Focus Array analysis. Raw data were analyzed by the Affymetrix NetAFFX Analysis Center online tools. These results provided by our Affymetrix data analysis were further analyzed through the use of Ingenuity Pathway Analysis software.

The nature of change of gene expression profile in response to each of the peptides consisting of the sequence RMPLPPRGCPAAAPWS (SEQ ID NO 10) or RPKHPIKHQGLPQEVLNENLLRF (SEQ ID NO 1) was determined. Four result files were generated: 1. Molecular networks; 2. Biological functions and diseases; 3. Metabolic-, signalling-, and other molecular pathways; 4. Network node molecule lists. Fischer's exact test was used to calculate a p-value determining the probability that each biological function and/or disease assigned to that data set is due to chance alone.

A number of genes of interest displayed altered expression in response to treatment with a peptide consisting of the sequence RMPLPPRGCPAAAPWS (SEQ ID NO 10). These include:
Amyloid Beta (A4) Precursor Protein (APP)—downregulated;
Stathmin-1—upregulated; and
protein-tyrosine phosphatase, receptor-type, F—downregulated.

A number of genes of interest displayed altered expression in response to treatment with a peptide consisting of the sequence RPKHPIKHQGLPQEVLNENLLRF (SEQ ID NO 1). These include:

Upregulation of bleomycin hydrolase;

glutamate receptors
Downregulation of glutamate receptor, ionotropic, N-methyl D-aspartate 2A;
Downregulation of glutamate receptor, metabotropic 7;
Downregulation of glutamate receptor, metabotropic 8;
Downregulation of brain glutamate decarboxylase 2;

Downregulation of Apolipoprotein E (APOE) and receptors.

Disease network analysis of gene expression profile was then carried out. Taking into consideration the nature of changes (up- and down-regulated genes), and based on the effect of each gene product, as well as their position in a specific network, the peptide was identified as having application in the prevention and/or treatment of several diseases.

The peptide of RMPLPPRGCPAAAPWS (SEQ ID NO 10) downregulates Amyloid Beta (A4) Precursor Protein (APP) gene expression, and thus it may have significance in prevention and treatment in central nervous system disorders such as dementia and Alzheimer's disease., and disorders related thereto. The peptide also upregulates Stathmin-1 expression, thus it may have therapeutic significance in the prevention and/or treatment of disorders characterised by uncontrolled cell growth and proliferation such as cancers, tumours and disorders related thereto. This peptide also downregulated protein-tyrosine phosphatase, receptor-type, F, thus it may have therapeutic significance in the prevention and/or treatment of disorders characterised by mutated protein-tyrosine phosphatase, receptor-type, F, and/or uncontrolled cell growth and proliferation such as cancers, tumours and disorders related thereto.

The peptide of sequence RPKHPIKHQGLPQEVLNENLLRF (SEQ ID NO 1) upregulates bleomycin hydrolase gene expression, and thus it may have significance in prevention and treatment in Alzheimer's disease. The peptide downregulates glutamate receptor expression, thus it could have therapeutic significance in Alzheimer's disease and other central nervous system disorders (e.g., epilepsy, amyotropic lateral sclerosis, Huntington's disease, ischemia and trauma). The peptide also down-regulates Apolipoprotein E, a major genetic risk factor for Alzheimer's disease, thus may have significance in prevention and treatment in Alzheimer's disease.

It will be appreciated that the invention described herein may be modified, within the scope of the claims.

In another embodiment, the present invention relates to peptides and their use in treating obesity and related disorders.

More than 65 percent of adults in the United States are overweight or obese. Obesity puts people at increased risk for chronic diseases, such as cardiovascular diseases, endocrine disorders, type II diabetes, high blood pressure, psychological disorders, respiratory disorders, stroke, infertility, osteoarthritis and several forms of cancer. Recent studies have associated a systemic inflammatory response characterized by endothelial cell dysfunction, oxidative stress, and circulating immune cell activation with obesity. For instance, adipocytes release a variety of cytokines, such as IL-1 and TNF-alpha, and cytokine-like substances, such as leptin and resistin, which appear to mediate this inflammatory response. The inflammatory response may be exacerbated by the insulin resistance that is often associated with obesity. Obesity also puts people at increased risk to several other diseases such as asthma, breast cancer and non-alcoholic liver steatosis. Thus elucidating the connections between obesity and these various diseases is crucial for understanding the role and function of adipose tissue and adipocytokines on the body, and in particular, the cardiovascular system, and may contribute towards designing of future therapeutic approaches. Adipocytokines such as leptin, adiponectin, resistin and visfatin are bioactive mediators, and have been implicated in the regulation of metabolism, energy storage and homeostasis. Adipocytokines are released from cells such as adipocytes present in adipose tissue, as well as other cells such as epithelial and the various lymphatic and inflammatory cells present within fat tissue. These bioactive mediators play a major role in the pathogenesis of a cluster of clinical symptoms such as insulin resistance, obesity, atherosclerosis, dyslipidemia and hypertension. Further research has shown that obesity may aggravate microvascular dysfunction associated with pathological states, such as sepsis.

Thus there is a need for new and effective treatments for preventing and/or treating obesity.

The present invention provides a peptide comprising the amino acid sequence FVAPFPEVFGKEKV (SEQ ID NO 2) (i.e. Phe-Val-Ala-Pro-Phe-Pro-Glu-Val-Phe-Gly-Lys-Glu-Lys-Val), or a salt thereof. Alternatively the present invention provides a peptide substantially consisting of the amino acid sequence FVAPFPEVFGKEKV (SEQ ID NO 2), or a salt thereof. Alternatively the present invention provides a peptide analogue of a peptide comprising the amino acid sequence FVAPFPEVFGKEKV (SEQ ID NO 2) in which one or more amino acids have been replaced, altered and/or deleted without substantially altering the biological properties of a peptide comprising the sequence FVAPFPEVFGKEKV (SEQ ID NO 2), or a salt thereof.

The present invention further provides a peptide comprising the amino acid sequence SDIPNPIGSENSEKTTMPLW (SEQ ID NO 3) (i.e. Ser-Asp-Ile-Pro-Asn-Pro-Ile-Gly-Ser-Glu-Asn-Ser-Glu-Lys-Thr-Thr-Met-Pro-Leu-Trp), or a salt thereof. Alternatively the present invention provides a peptide substantially consisting of the amino acid sequence SDIPNPIGSENSEKTTMPLW (SEQ ID NO 3), or a salt thereof. Alternatively the present invention provides a peptide analogue of a peptide comprising the amino acid sequence SDIPNPIGSENSEKTTMPLW (SEQ ID NO 3) in which one or more amino acids have been replaced, altered and/or deleted without substantially altering the biological properties of a peptide comprising the sequence SDIPNPIGSENSEKTTMPLW (SEQ ID NO 3), or a salt thereof.

The present invention further provides a peptide comprising the amino acid sequence GPVRGPFPI (SEQ ID NO 4) (i.e. Gly-Pro-Val-Arg-Gly-Pro-Phe-Pro-Ile), or a salt thereof. Alternatively the present invention provides a peptide substantially consisting of the amino acid sequence GPVRGPFPI (SEQ ID NO 4), or a salt thereof. Alternatively the present invention provides a peptide analogue of a peptide comprising the amino acid sequence GPVRGPFPI (SEQ ID NO 4) in which one or more amino acids have been replaced, altered and/or deleted without substantially altering the biological properties of a peptide comprising the sequence GPVRGPFPI (SEQ ID NO 4), or a salt thereof.

The present invention further provides a peptide comprising the amino acid sequence EPVLGPVRGPFPI (SEQ ID NO 5) (i.e. Glu-Pro-Val-Leu-Gly-Pro-Val-Arg-Gly-Pro-Phe-Pro-Ile), or a salt thereof. Alternatively the present invention provides a peptide substantially consisting of the amino acid sequence EPVLGPVRGPFPI (SEQ ID NO 5), or a salt thereof. Alternatively the present invention provides a peptide analogue of a peptide comprising the amino acid sequence EPVLGPVRGPFPI (SEQ ID NO 5) in which one or more amino acids have been replaced, altered and/or deleted without substantially altering the biological properties of a peptide comprising the sequence EPVLGPVRGPFPI (SEQ ID NO 5), or a salt thereof.

The present invention further provides a peptide comprising the amino acid sequence VPYPQRDMPIQ (SEQ ID NO 6) (i.e. Val-Pro-Tyr-Pro-Gln-Arg-Asp-Met-Pro-Ile-Gln), or a salt thereof. Alternatively the present invention provides a peptide substantially consisting of the amino acid sequence VPYPQRDMPIQ (SEQ ID NO 6), or a salt thereof. Alternatively the present invention provides a peptide analogue of a peptide comprising the amino acid sequence VPYPQRDMPIQ (SEQ ID NO 6) in which one or more amino acids have been replaced, altered and/or deleted without substantially altering the biological properties of a peptide comprising the sequence VPYPQRDMPIQ (SEQ ID NO 6), or a salt thereof.

The present invention further provides a peptide comprising the amino acid sequence SLSQSKVLPVPQKAVPYPQRDMPIQ (SEQ ID NO 7) (i.e. Ser-Leu-Ser-Gln-Ser-Lys-Val-Leu-Pro-Val-Pro-Gln-Lys-Ala-Val-Pro-Tyr-Pro-Gln-Arg-Asp-Met-Pro-Ile-Gln), or a salt thereof. Alternatively the present invention provides a peptide substantially consisting of the amino acid sequence SLSQSKVLPVPQKAVPYPQRDMPIQ (SEQ ID NO 7), or a salt thereof. Alternatively the present invention provides a peptide analogue of a peptide comprising the amino acid sequence SLSQSKVLPVPQKAVPYPQRDMPIQ (SEQ ID NO 7) in which one or more amino acids have been replaced, altered and/or deleted without substantially altering the biological properties of a peptide comprising the sequence SLSQSKVLPVPQKAVPYPQRDMPIQ (SEQ ID NO 7), or a salt thereof.

The present invention further provides a peptide comprising the amino acid sequence EPVLGPVR (SEQ ID NO 8) (i.e. Glu-Pro-Val-Leu-Gly-Pro-Val-Arg), or a salt thereof. Alternatively the present invention provides a peptide substantially consisting of the amino acid sequence EPVLGPVR (SEQ ID NO 8), or a salt thereof. Alternatively the present invention provides a peptide analogue of a peptide comprising the amino acid sequence EPVLGPVR (SEQ ID NO 8) in which one or more amino acids have been replaced, altered and/or deleted without substantially altering the biological properties of a peptide comprising the sequence EPVLGPVR (SEQ ID NO 8), or a salt thereof.

The present invention further provides a composition comprising 2 or more peptides, wherein each peptide is different, wherein the peptides are selected from a list comprising: a peptide comprising the amino acid sequence RPKHPIKHQGLPQEVLNENLLRF (SEQ ID NO 1) (i.e. Arg-Pro-Lys-His-Pro-Ile-Lys-His-Gln-Gly-Leu-Pro-Gln-Glu-Val-Leu-Asn-Glu-Asn-Leu-Leu-Arg-Phe), or a salt thereof; a peptide substantially consisting of the amino acid sequence RPKHPIKHQGLPQEVLNENLLRF (SEQ ID NO 1), or a salt thereof; a peptide analogue of a peptide comprising the amino acid sequence RPKHPIKHQGLPQEVLNENLLRF (SEQ ID NO 1) in which one or more amino acids have been replaced, altered and/or deleted without substantially altering the biological properties of a peptide comprising the sequence RPKHPIKHQGLPQEVLNENLLRF (SEQ ID NO 1), or a salt thereof; a peptide comprising any one of the amino acid sequences of SEQ ID NO 2, 3, 4, 5, 6, 7 and 8, or a salt thereof; a peptide substantially consisting of any of one the amino acid sequences SEQ ID NO 2, 3, 4, 5, 6, 7 and 8, or a salt thereof; and a peptide analogue of a peptide comprising any one of the amino acid sequences SEQ ID NO 2, 3, 4, 5, 6, 7 and 8, in which one or more amino acids have been replaced, altered and/or deleted without substantially altering the biological properties of a peptide comprising the amino acid sequence SEQ ID NO 2, 3, 4, 5, 6, 7 and 8, respectively, or a salt thereof.

The amino acid sequence FVAPFPEVFGKEKV (SEQ ID NO 2) is homologous with the amino acid sequence, positions 39 to 52, of casein alpha S-1.

The amino acid sequence SDIPNPIGSENSEKTTMPLW (SEQ ID NO 3) is homologous with the amino acid sequence, positions 195-214, of casein alpha S-1.

The amino acid sequence GPVRGPFPI (SEQ ID NO 4) is homologous with the amino acid sequence, positions 199-207, of casein beta.

The amino acid sequence EPVLGPVRGPFPI (SEQ ID NO 5) is homologous with the amino acid sequence, positions 195-207, of casein beta.

The amino acid sequence VPYPQRDMPIQ (SEQ ID NO 6) is homologous with the amino acid sequence, positions 178-188, of casein beta.

The amino acid sequence SLSQSKVLPVPQKAVPYPQRDMPIQ (SEQ ID NO 7) is homologous with the amino acid sequence, positions 164-188, of casein beta.

The amino acid sequence EPVLGPVR (SEQ ID NO 8) is homologous with the amino acid sequence, positions 195-202, of casein beta.

The amino acid sequence RPKHPIKHQGLPQEVLNENLLRF (SEQ ID NO 1) is homologous with the amino acid sequence, positions 16-38, of casein alpha S-1.

The peptides according to the present invention have a number of therapeutic uses. In particular, these peptides have been found to be useful in the prevention and treatment of obesity. It has been found that the peptides according to the present invention upregulate the gene expression and protein expression of leptin. Thus, in accordance with the present invention, there are provided peptides for upregulating the gene expression and protein expression of leptin in a cell.

Leptin is a 167 amino-acid protein hormone, encoded by the ob gene, and plays a key role in regulating appetite, metabolism and energy expenditure. Leptin is primarily produced by adipocytes, but it was also shown to be generated by gastric epithelial cells, endothelial cells, placenta, ovary, skeletal muscle and liver. Neurons containing Neuropeptide Y (NPY) are involved in increasing food intake; by inhibiting the activity of these neurons, leptin induces a sense of satiety, thus decreasing food intake. Leptin also stimulates neurons expressing α-Melanocyte-Stimulating Hormone (α-MSH), which hormone is also involved in the sensation of satiety. Agouti-Related Peptide (AgRP), which increases appetite and decreases metabolism, is another neuropeptide whose activity is inhibited by leptin.

Homozygous mutations in the leptin gene result in to hyperphagy and severe obesity. This condition can be treated by the administration of recombinant leptin.

Leptin exerts its effects, inter alia, by binding with leptin receptors present on ventral medial nucleus of the hypothalamus, which induces a sensation of satiety. Leptin is thought to change expression levels of endocannabinoids in cells, associated with increasing appetite. Mice with mutated ob gene (ob/ob mice) develop obesity in relation to the lack of satiety signalling within their brain gut axis. Adult animals with leptin deficiency show increased appetite and obesity which can be treated by leptin. These animals also exhibit T cell hypo-responsiveness, hyperinsulinemia and insulin resistance, hyperlipidemia, immune dysfunction, and neuroendocrine abnormalities.

Plasma levels of leptin in humans are closely associated with the fat mass. Like the majority of neurohormones, leptin levels exhibit important circadian rhythms. Several agonists including TNF-alpha and other pro-inflammatory cytokines, insulin, glucose, and estrogens have been shown to increase leptin release from adipocytes. Increased levels of other vasoactive factors like angiotensin II or endothelin may also lead to leptin generation, although this phenomenon may occur locally since it does not seem to affect plasma levels of leptin during angiotensin II administration. Leptin receptors are widely expressed on various cells including cells of the cardiovascular and immune system. Thus, upregulation of gene and/or protein expression of leptin may increase the amount of leptin in a cell. Alternatively, upregulation of gene and/or protein expression of leptin may increase the amount of leptin circulating in the body. Alternatively, upregulation of gene and/or protein expression of leptin may inhibit the activity of any one of Neuropeptide Y (NPY), Agouti-Related Peptide (AgRP) and α-Melanocyte-Stimulating Hormone (α-MSH), in a cell. Alternatively, upregulation of gene and/or protein expression of leptin may change the expression levels of endocannabinoids in a cell, or more particularly, decrease the expression levels of endocannabinoids in a cell. Alternatively, upregulation of gene and/or protein expression of leptin may increase the sensation of satiety. Alternatively, upregulation of gene and/or protein expression of leptin may decrease appetite. Alternatively, upregulation of gene and/or protein expression of leptin may increase the metabolic rate, of a cell. Alternatively, upregulation of gene and/or protein expression of leptin may increase the energy expenditure of a cell. Thus, advantageously, the peptides in accordance with the present invention may be used to prevent and/or treat obesity. Thus, advantageously, the peptides in accordance with the present invention may be used to prevent and/or treat hyperphagy. Thus, advantageously, the peptides in accordance with the present invention may be used to prevent and/or treat an eating disorder. Thus, advantageously, the peptides in accordance with the present invention may be used to prevent and/or treat disorders characterised by a low metabolism and/or energy expenditure.

In addition to the foregoing, the peptides of the present invention, or salts thereof, may be used to prevent and/or treat leptin associated disorders, including anorexia, increased body fat deposition, hyperglycemia, hyperinsulinemia, hypothermia, hypophagia, impaired thyroid and reproductive function (in both men and women), obesity-related dysfunctions (e.g. type II diabetes mellitus), as well as obesity-associated clinical and psychological morbidities, including hypertension, elevated blood lipids, and decreased life expectancy. Other leptin-related disorders include severe morning sickness, polycystic ovary syndrome, bone growth, aberrant T-cell activity in response to atherosclerosis, and angiogenesis (increased VEGF levels). The peptides of the present invention, or salts thereof, may also be used to control body mass (i.e. weight), and may also be used in the modulation of amount of body fat, or the modulation of energy expenditure or metabolism of a patient.

In addition to the foregoing, the peptides of the present invention, or salts thereof, may be used to prevent and/or treat obesity-related and obesity-associated disorders and disorders related to type II diabetes mellitus such as hyperlipidemia; dyslipidemia; abdominal obesity; hypercholesterolemia; hypertrigyceridemia; atherosclerosis; coronary heart disease; stroke; hypertension; peripheral vascular disease; vascular restenosis; nephropathy; neuropathy; inflammatory conditions, such as, but not limited to, irritable bowel syndrome, inflammatory bowel disease, including Crohn's disease and ulcerative colitis; other inflammatory conditions; pancreatitis; neurodegenerative disease; retinopathy; neoplastic conditions, such as, but not limited to adipose cell tumours, adipose cell carcinomas, such as liposarcoma; cancers, including gastric and bladder cancers; angiogenesis; Alzheimer's disease; psoriasis; and other disorders where insulin resistance is a component. The peptides of the invention may also be useful in the treatment, control and/or prevention of overeating; bulimia; elevated plasma insulin concentrations; insulin resistance; glucose tolerance; Metabolic Syndrome; lipid disorders; low HDL levels; diabetes while mitigating cardiac hypertrophy, including left ventricular hypertrophy; high LDL levels; hyperglycemia; neoplastic conditions, such as endometrial, breast, prostate, kidney and colon cancer; osteoarthritis; obstructive sleep apnea; gallstones; abnormal heart rhythms; heart arrythmias; myocardial infarction; congestive heart failure; sudden death; ovarian hyperandrogenism, (polycystic ovary disease); craniopharyngioma; the Prader-Willi Syndrome; Frohlich's syndrome; GH-deficient subjects; normal variant short stature; Turner's syndrome; and other pathological conditions showing reduced metabolic activity or a decrease in resting energy expenditure as a percentage of total fat-free mass, e.g., children with acute lymphoblastic leukemia.

The peptides in accordance with the present invention, when administered in combination to a patient suffering from a disorder such as obesity, or any other leptin-associated disorder, may affect the body physiologically in a synergistic manner, such that the physiological effect of the combination is greater than the sum of the physiological effect of each of the peptides when administered alone. The synergistic effect of the combinations of the peptides of the present invention may result from the combination of effects of the peptides on different physiological targets. The synergy of the combined beneficial physiological response resulting from the administration of the combinations of peptides in accordance with the present invention may result in an improvement in the treatment and/or prevention of a disorder such as obesity, or any other leptin-associated disorder, compared to administration of each peptide separately.

The peptides of the present invention may be provided in substantially isolated and/or purified form from a natural source. Alternatively, they may be formed by a synthetic process.

For the avoidance of doubt, it is stated that the amino-terminal end is on the left hand side of the sequence, in accordance with the usual convention. Alternatively, sequences may be annotated specifying the N-terminal and C-terminal ends. Thus, for instance, amino acid sequence SEQ ID NO 2 may be annotated as NH₂-FVAPFPEVFGKEKV-COOH, amino acid sequence SEQ ID NO 3 may be annotated as NH₂-SDIPNPIGSENSEKTTMPLW-COOH, and so on.

It will be appreciated that the specified amino acid sequences may be provided with an inert amino acid sequence on the amino-terminal and/or the carboxy-terminal end thereof. The inert amino acid sequence may be a single amino acid, or a peptide containing between 2 and 5 amino acids, or a peptide containing 2 to 10 amino acids. It will be appreciated by a person skilled in the art that these inert sequences do not substantially contribute to or change the biological properties of the specified amino acid sequences, i.e. any one of SEQ ID NO 1, 2, 3, 4, 5, 6, 7 or 8. Furthermore, it will be appreciated by a person skilled in the art that the inert amino acid sequences may be varied. Furthermore it will be appreciated that certain inert sequences may be unsuitable. For instance, if a single alanine residue is provided at one terminal end of the specified amino acid sequence, then the skilled person will recognise that the provision of a glycine residue at the other terminal end of the peptide will be unsuitable.

The present invention is also directed to peptides that are polymorphs, homologues (preferably mammalian) and physiologically acceptable active derivatives of the peptides of SEQ ID NO 1, 2, 3, 4, 5, 6, 7 and 8, including salts thereof, which have substantially the same biological properties of the peptide of SEQ ID NO 1, 2, 3, 4, 5, 6, 7 and 8, respectively. These polymorphs, homologues and physiologically acceptable active derivatives may bind to antibodies (either monoclonal or polyclonal) raised against a peptide comprising or consisting of the amino acid sequence of SEQ ID NO 1, 2, 3, 4, 5, 6, 7 and 8, respectively, and conservatively modified peptide analogues thereof, or may have substantial sequence identity (i.e. at least about 60%) to a peptide consisting of the amino acid sequence of SEQ ID NO 1, 2, 3, 4, 5, 6, 7 and 8, respectively, and conservatively modified peptide analogues thereof.

The term percent sequence identity refers to two or more sequences that are the same or have a specified percentage of amino acid residues that are the same, when aligned for maximum correspondence over a comparison window, in accordance with techniques well known to a person skilled in the art. For example, an amino acid sequence identity of 60% refers to sequences that have at least about 60% amino acid identity when aligned for maximum correspondence over a comparison window in accordance with techniques known to a person skilled in the art. Preferably the sequence identity is about 60%, more preferably 60-70%, more preferably 70-80%, more preferably 80-90%, more preferably about or greater than 90%.

In accordance with techniques well known to the person skilled in the art, it will be recognized that amino acid positions that are not identical may differ by conservative amino acid substitutions, where amino acids residues are substituted for other amino acid residues with similar chemical properties (e.g. size, charge and/or hydrophobicity). Conservative amino acid substitutions generally do not greatly affect the biological properties of the peptide. Examples of conservative amino acid substitutions include substitution of leucine with isoleucine, and substitution of serine with threonine. Examples of non-conservative substitutions include substitution of aspartic acid with lysine, and substitution of glycine with tryptophan.

Where sequences differ in conservative amino acid substitutions, the sequence identity may be corrected to take account for the conservative nature of the amino acid substitution. Means for making this adjustment are well known to those of skilled in the art. For instance, a conservative substitution would be scored as a partial rather than a full mismatch, and thus a conservative substitution would increase the percentage sequence identity compared to a non-conservative substitution. Thus, for example, when comparing two amino acid sequences, where an identical amino acid is given a score of 1 and a non-conservative substitution is given a score of zero, a conservative substitution may be given a score between zero and 1. Techniques of scoring conservative substitutions for the purposes of determining percentage sequence identity are well known to the person skilled in the art.

The peptides may be obtained by a number of techniques. In one embodiment, it is prepared by a conventional technique for peptide synthesis, such as by solid-phase or liquid-phase peptide synthesis. Alternatively, the gene sequence encoding the peptide can be constructed by known techniques, inserted into expression vectors or plasmids, and transfected into suitable microorganisms that will express the DNA translated sequences as the peptide, whereby the peptide can be later extracted from the medium in which the microorganisms are grown.

A chronic disorder is a disorder that has persisted, or is expected to persist, for a long time, i.e., at least 3 months and usually at least 6 months.

The peptides also have diagnostic and research applications. For example, a synthetic peptide of any one of SEQ ID NO 1, 2, 3, 4, 5, 6, 7 and 8, as well as the corresponding antibodies described below, may be used to recognise pathological processes occurring in a host. These processes may be induced by excessive production or inhibition of the peptide or the antibodies. Once the pathological process associated with a particular level of the peptide or the antibodies is known, measuring the production of the peptide and the antibodies in body fluids may be used to determine pathological processes taking place in the host. This may occur, for example, in lactating mothers during various infections or drug treatments.

According to another aspect of the invention, we provide the use of the peptides as a dietary supplement. This dietary supplement may be particularly useful for obese patients, or with patients suffering from a leptin-related disorder. In an aspect of the invention, we provide a dietary supplement comprising an orally ingestible blend of the peptides in combination with a physiologically acceptable carrier. The dietary supplement may be provided in liquid or solid form; the dietary supplement may suitably be provided in the form of a tablet. The dietary supplement may be provided in the form of a baby food formula. The dietary supplement may include, as an additive, lactoferrin and/or selenium and/or a group of cytokines containing members of the interferon family.

The peptides of the invention may be administered prophylactically in order to help to prevent the development of obesity, or any other leptin-related disorder.

The peptides in accordance with the invention may be administered in a dosage in the range 1 nM to 10 mM. A dosage unit of about 2 μM is typical. However, the optimum dosage will, of course, depend upon the condition being treated.

Each peptide of the combination of peptides in accordance with the present invention may be administered simultaneously or sequentially, in any therapeutically appropriate combination.

The combination of peptides may be formulated and administered as a single pharmaceutical composition comprising the peptides.

Alternatively each peptide may be formulated as separate compositions, which may then be administered simultaneously.

Alternatively the peptides of the combination may be administered sequentially. Preferably each peptide of the combination is administered within a biologically relevant time frame. In other words, one peptide of the combination is administered during the period when a patient is experiencing the biological effects of the administration of another peptide of the combination.

Each peptide of the combination is preferably present in equimolar ratios. However, it will be appreciated by the person skilled in the art that the ratios of the peptides in the combination may be varied. For instance, when two peptides are present in the combination, the molar ratios of one peptide to the other may vary from 1:1000 to 1000:1, more preferably 1:100 to 100:1, more preferably 1:10 to 10:1, more preferably 1:5 to 5:1, more preferably 1:4 to 4:1, more preferably 1:3 to 3:1, more preferably 1:2 to 2:1. The skilled person will appreciate other ratios may be desirable, depending on the therapeutic application. When there are 3 or more peptides present in the combination, they are preferably present in equimolar amounts. Alternatively, the relative amounts of each peptide may vary, depending on the therapeutic application.

The peptides in accordance with the invention may be formulated for administration in any suitable form. Thus, the use in accordance with the invention may be in the form of a composition, especially a pharmaceutical composition, which includes the peptide in combination with a physiologically acceptable carrier. The peptide may, for example, be formulated for oral, topical, rectal or parenteral administration. More specifically, the peptide may be formulated for administration by injection, or, preferably, in a form suitable for absorption through the mucosa of the oral/nasopharyngeal cavity, the alimentary canal or any other mucosal surface. The peptide may be formulated for administration intravenously, subcutaneously, or intramuscularly. The oral formulations may be provided in a form for swallowing or, preferably, in a form for dissolving in the saliva, whereby the formulation can be absorbed in the mucous membranes of the oral/nasopharyngeal cavity. The oral formulations may be in the form of a tablet (i.e. fast dissolving tablets) for oral administration, lozenges (i.e. a sweet-like tablet in a form suitable to be retained in the mouth and sucked), or adhesive gels for rubbing into the gum. The peptide may be formulated as an adhesive plaster or patch, which may be applied to the gums. The peptide may also be formulated for application to mucous-membranes of the genito-urinary organs. The topical formulations may be provided in the form of, for example, a cream or a gel. The peptide may also be formulated as a spray for application to the nasopharyngeal or bronchial mucous surface.

The peptides may be incorporated into products like milk, yogurts, milkshake, ice cream, cheese spread and various beverage products, including sport drinks.

In another aspect, the invention provides an antibody for the peptides, and provides compositions containing said antibodies. In particular the invention provides the antibodies in substantially isolated form. The antibodies can be produced by injecting a suitable subject, such as a rabbit, with the peptides (with a suitable adjuvant), then recovering the antibodies from the subject after allowing time for them to be produced. It is possible to test that the correct antibody has been produced by ELISA (enzyme-linked immunosorbent assay) using the synthetic peptide as antigens. The antibodies have potential uses in therapy, as a diagnostic tool and as a research tool. The antibodies can be produced in accordance with the methods described in example 3 of WO00/75173.

The invention also encompasses the selective administration of the peptides, at selected times to a patient.

In some applications it may be desirable to provide a pharmaceutical composition which contains the peptides in combination with a physiologically acceptable carrier.

The invention further embraces the use of the peptides in the manufacture of a medicament for use in any of the therapeutic applications described above.

The invention further embraces the methods of treating a mammal, in particular human, in any of the therapeutic applications described above.

### EXAMPLE 1

TR146 buccal mucosal cells, obtained from Cancer Research U.K., were propagated in Dulbecco's modified Eagle medium (DMEM) high glucose (Gibco). The culture medium supplemented with 3.7 mg/ml NaHCO₃, 10% FCS, 50 units/ml penicillin G, and 50 mg/ml streptomycin sulphate. Cells were passaged when 90% confluence was reached. The medium was discarded, and cells were washed twice with sterile DPBS (without calcium and magnesium, Cellgro) and 0.25% trypsin-EDTA solution (Gibco) was added. The flask was placed at 37° C. for 10 minutes and then detached cells were suspended in growth medium and seeded in new flasks (dishes micro-well plates).

### EXAMPLE 2

For proteomic microarray analysis, cells at 75-80% confluence in T75 flasks were treated with equimolar (2.0 μM) concentrations of a peptide consisting of the sequence FVAPFPEVFGKEKV (SEQ ID NO 2), SDIPNPIGSENSEKTTMPLW (SEQ ID NO 3), GPVRGPFPI (SEQ ID NO 4), EPVLGPVRGPFPI (SEQ ID NO 5), VPYPQRDMPIQ (SEQ ID NO 6), SLSQSKVLPVPQKAVPYPQRDMPIQ (SEQ ID NO 7), EPVLGPVR (SEQ ID NO 8) and RPKHPIKHQGLPQEVLNENLLRF (SEQ ID NO 1). Mock-treated cells received same volume of solvent. After 24 hours the cells were washed twice with DPBS, trypsinized, suspended in 5 ml growth media and centrifuged (800 g for 10 minutes). Cells were resuspended in 5 ml DPBS and centrifuged again.

### EXAMPLE 3

Antibodies specific to leptin were immobilised on the surface of a membrane. Sample extracts of cell samples each treated with peptides of amino acid sequences SEQ ID NO 1, 2, 3, 4, 5, 6, 7 and 8 were incubated with the membranes. Biotinylated antibodies, specific to leptin-antibody complexes, were then incubated with the membranes. Bound leptin was quantified by measuring chemiluminescence.

Protein expression of leptin was upregulated in TR146 buccal mucosal cells in response to treatment with each of the peptides of SEQ ID NO 1, 2, 3, 4, 5, 6, 7 and 8.

Thus the peptides of SEQ ID NO 1, 2, 3, 4, 5, 6, 7 and 8, alone or in combination, may have significance in the prevention and/or treatment of obesity, disorders associated with obesity, such as hyperphagy and type II diabetes mellitus, and leptin-associated disorders. The peptides may also be used to control body mass.

It will be appreciated that the invention described herein may be modified, within the scope of the claims.

In another embodiment the present invention relates to peptides and their use in treating obesity and related disorders.

The present invention provides a peptide comprising the amino acid sequence FVAPFPEVFGKEKV (SEQ ID NO 2) (i.e. Phe-Val-Ala-Pro-Phe-Pro-Glu-Val-Phe-Gly-Lys-Glu-Lys-Val), or a salt thereof. Alternatively the present invention provides a peptide substantially consisting of the amino acid sequence FVAPFPEVFGKEKV (SEQ ID NO 2), or a salt thereof. Alternatively the present invention provides a peptide analogue of a peptide comprising the amino acid sequence FVAPFPEVFGKEKV (SEQ ID NO 2) in which one or more amino acids have been replaced, altered and/or deleted without substantially altering the biological properties of a peptide comprising the sequence FVAPFPEVFGKEKV (SEQ ID NO 2), or a salt thereof.

The present invention further provides a peptide comprising the amino acid sequence RGPFPIIV (SEQ ID NO 9) (i.e. Arg-Gly-Pro-Phe-Pro-Ile-Ile-Val), or a salt thereof. Alternatively the present invention provides a peptide substantially consisting of the amino acid sequence RGPFPIIV (SEQ ID NO 9), or a salt thereof. Alternatively the present invention provides a peptide analogue of a peptide comprising the amino acid sequence RGPFPIIV (SEQ ID NO 9) in which one or more amino acids have been replaced, altered and/or deleted without substantially altering the biological properties of a peptide comprising the sequence RGPFPIIV (SEQ ID NO 9), or a salt thereof.

The present invention further provides a peptide comprising the amino acid sequence RMPLPPRGCPAAAPWS (SEQ ID NO 10) (i.e. Arg-Met-Pro-Leu-Pro-Pro-Arg-Gly-Cys-Pro-Ala-Ala-Ala-Pro-Trp-Ser), or a salt thereof. Alternatively the present invention provides a peptide substantially consisting of the amino acid sequence RMPLPPRGCPAAAPWS (SEQ ID NO 10), or a salt thereof. Alternatively the present invention provides a peptide analogue of a peptide comprising the amino acid sequence RMPLPPRGCPAAAPWS (SEQ ID NO 10) in which one or more amino acids have been replaced, altered and/or deleted without substantially altering the biological properties of a peptide comprising the sequence RMPLPPRGCPAAAPWS (SEQ ID NO 10), or a salt thereof.

The present invention further provides a composition comprising 2 or more peptides, wherein each peptide is different, wherein the peptides are selected from a list comprising: a peptide comprising any one of the amino acid sequences of SEQ ID NO 2, 9 and 10, or a salt thereof; a peptide substantially consisting of any of one the amino acid sequences SEQ ID NO 2, 9 and 10, or a salt thereof; and a peptide analogue of a peptide comprising any one of the amino acid sequences SEQ ID NO 2, 9 and 10, in which one or more amino acids have been replaced, altered and/or deleted without substantially altering the biological properties of a peptide comprising the amino acid sequence SEQ ID NO 2, 9 and 10, respectively, or a salt thereof.

The amino acid sequence RGPFPIIV (SEQ ID NO 9) is homologous with the amino acid sequence, positions 202-209, of casein alpha S-1.

The peptides according to the present invention have a number of therapeutic uses. In particular, these peptides have been found to be useful in the prevention and treatment of obesity.

In addition to the foregoing, the peptides of the present invention, or salts thereof, may be used to prevent and/or treat obesity-related and obesity-associated disorders and disorders related to type II diabetes mellitus such as hyperlipidemia; dyslipidemia; abdominal obesity; hypercholesterolemia; hypertrigyceridemia; atherosclerosis; coronary heart disease; stroke; hypertension; peripheral vascular disease; vascular restenosis; nephropathy; neuropathy; inflammatory conditions, such as, but not limited to, irritable bowel syndrome, inflammatory bowel disease, including Crohn's disease and ulcerative colitis; other inflammatory conditions; pancreatitis; neurodegenerative disease; retinopathy; neoplastic conditions, such as, but not limited to adipose cell tumours, adipose cell carcinomas, such as liposarcoma; cancers, including gastric and bladder cancers; angiogenesis; Alzheimer's disease; psoriasis; and other disorders where insulin resistance is a component. The peptides of the invention may also be useful in the treatment, control and/or prevention of overeating; bulimia; elevated plasma insulin concentrations; insulin resistance; glucose tolerance; Metabolic Syndrome; lipid disorders; low HDL levels; diabetes while mitigating cardiac hypertrophy, including left ventricular hypertrophy; high LDL levels; hyperglycemia; neoplastic conditions, such as endometrial, breast, prostate, kidney and colon cancer; osteoarthritis; obstructive sleep apnea; gallstones; abnormal heart rhythms; heart arrythmias; myocardial infarction; congestive heart failure; sudden death; ovarian hyperandrogenism, (polycystic ovary disease); craniopharyngioma; the Prader-Willi Syndrome; Frohlich's syndrome; GH-deficient subjects; normal variant short stature; Turner's syndrome; and other pathological conditions showing reduced metabolic activity or a decrease in resting energy expenditure as a percentage of total fat-free mass, e.g., children with acute lymphoblastic leukemia.

In addition to the foregoing, the peptides of the present invention, or salts thereof, may be used to prevent and/or treat leptin associated disorders, including anorexia, increased body fat deposition, hyperglycemia, hyperinsulinemia, hypothermia, hypophagia, impaired thyroid and reproductive function (in both men and women), obesity-related dysfunctions (e.g. type II diabetes mellitus), as well as obesity-associated clinical and psychological morbidities, including hypertension, elevated blood lipids, and decreased life expectancy. Other leptin-related disorders include severe morning sickness, polycystic ovary syndrome, bone growth, aberrant T-cell activity in response to atherosclerosis, and angiogenesis (increased VEGF levels). The peptides of the present invention, or salts thereof, may also be used to control body mass (i.e. weight), and may also be used in the modulation of amount of body fat, or the modulation of energy expenditure or metabolism of a patient.

The peptides in accordance with the present invention, when administered in combination to a patient suffering from a disorder such as obesity, or any other leptin-associated disorder, may affect the body physiologically in a synergistic manner, such that the physiological effect of the combination is greater than the sum of the physiological effect of each of the peptides when administered alone. The synergistic effect of the combinations of the peptides of the present invention may result from the combination of effects of the peptides on different physiological targets. The synergy of the combined beneficial physiological response resulting from the administration of the combinations of peptides in accordance with the present invention may result in an improvement in the treatment and/or prevention of a disorder such as obesity, or any other leptin-associated disorder, compared to administration of each peptide separately.

The peptides of the present invention may be provided in substantially isolated and/or purified form from a natural source. Alternatively, they may be formed by a synthetic process.

For the avoidance of doubt, it is stated that the amino-terminal end is on the left hand side of the sequence, in accordance with the usual convention. Alternatively, sequences may be annotated specifying the N-terminal and C-terminal ends. Thus, for instance, amino acid sequence SEQ ID NO 2 may be annotated as NH₂-FVAPFPEVFGKEKV-COOH, amino acid sequence SEQ ID NO 3 may be annotated as NH₂-SDIPNPIGSENSEKTTMPLW-COOH, and so on.

It will be appreciated that the specified amino acid sequences may be provided with an inert amino acid sequence on the amino-terminal and/or the carboxy-terminal end thereof. The inert amino acid sequence may be a single amino acid, or a peptide containing between 2 and 5 amino acids, or a peptide containing 2 to 10 amino acids. It will be appreciated by a person skilled in the art that these inert sequences do not substantially contribute to or change the biological properties of the specified amino acid sequences, i.e. any one of SEQ ID NO 2, 9, or 10. Furthermore, it will be appreciated by a person skilled in the art that the inert amino acid sequences may be varied. Furthermore it will be appreciated that certain inert sequences may be unsuitable. For instance, if a single alanine residue is provided at one terminal end of the specified amino acid sequence, then the skilled person will recognise that the provision of a glycine residue at the other terminal end of the peptide will be unsuitable.

The present invention is also directed to peptides that are polymorphs, homologues (preferably mammalian) and physiologically acceptable active derivatives of the peptides of SEQ ID NO 2, 9, or 10, including salts thereof, which have substantially the same biological properties of the peptide of SEQ ID NO 2, 9, or 10, respectively. These polymorphs, homologues and physiologically acceptable active derivatives may bind to antibodies (either monoclonal or polyclonal) raised against a peptide comprising or consisting of the amino acid sequence of SEQ ID NO 2, 9, or 10, respectively, and conservatively modified peptide analogues thereof, or may have substantial sequence identity (i.e. at least about 60%) to a peptide consisting of the amino acid sequence of SEQ ID NO 2, 9, or 10, respectively, and conservatively modified peptide analogues thereof.

The term percent sequence identity refers to two or more sequences that are the same or have a specified percentage of amino acid residues that are the same, when aligned for maximum correspondence over a comparison window, in accordance with techniques well known to a person skilled in the art. For example, an amino acid sequence identity of 60% refers to sequences that have at least about 60% amino acid identity when aligned for maximum correspondence over a comparison window in accordance with techniques known to a person skilled in the art. Preferably the sequence identity is about 60%, more preferably 60-70%, more preferably 70-80%, more preferably 80-90%, more preferably about or greater than 90%.

In accordance with techniques well known to the person skilled in the art, it will be recognized that amino acid positions that are not identical may differ by conservative amino acid substitutions, where amino acids residues are substituted for other amino acid residues with similar chemical properties (e.g. size, charge and/or hydrophobicity). Conservative amino acid substitutions generally do not greatly affect the biological properties of the peptide. Examples of conservative amino acid substitutions include substitution of leucine with isoleucine, and substitution of serine with threonine. Examples of non-conservative substitutions include substitution of aspartic acid with lysine, and substitution of glycine with tryptophan.

Where sequences differ in conservative amino acid substitutions, the sequence identity may be corrected to take account for the conservative nature of the amino acid substitution. Means for making this adjustment are well known to those of skilled in the art. For instance, a conservative substitution would be scored as a partial rather than a full mismatch, and thus a conservative substitution would increase the percentage sequence identity compared to a non-conservative substitution. Thus, for example, when comparing two amino acid sequences, where an identical amino acid is given a score of 1 and a non-conservative substitution is given a score of zero, a conservative substitution may be given a score between zero and 1. Techniques of scoring conservative substitutions for the purposes of determining percentage sequence identity are well known to the person skilled in the art.

The peptides may be obtained by a number of techniques. In one embodiment, it is prepared by a conventional technique for peptide synthesis, such as by solid-phase or liquid-phase peptide synthesis. Alternatively, the gene sequence encoding the peptide can be constructed by known techniques, inserted into expression vectors or plasmids, and transfected into suitable microorganisms that will express the DNA translated sequences as the peptide, whereby the peptide can be later extracted from the medium in which the microorganisms are grown.

A chronic disorder is a disorder that has persisted, or is expected to persist, for a long time, i.e., at least 3 months and usually at least 6 months.

The peptides also have diagnostic and research applications. For example, a synthetic peptide of any one of SEQ ID NO 2, 9, or 10, as well as the corresponding antibodies described below, may be used to recognise pathological processes occurring in a host. These processes may be induced by excessive production or inhibition of the peptide or the antibodies. Once the pathological process associated with a particular level of the peptide or the antibodies is known, measuring the production of the peptide and the antibodies in body fluids may be used to determine pathological processes taking place in the host. This may occur, for example, in lactating mothers during various infections or drug treatments.

According to another aspect of the invention, we provide the use of the peptides as a dietary supplement. This dietary supplement may be particularly useful for obese patients, or with patients suffering from a leptin-related disorder. In an aspect of the invention, we provide a dietary supplement comprising an orally ingestible blend of the peptides in combination with a physiologically acceptable carrier. The dietary supplement may be provided in liquid or solid form; the dietary supplement may suitably be provided in the form of a tablet. The dietary supplement may be provided in the form of a baby food formula. The dietary supplement may include, as an additive, lactoferrin and/or selenium and/or a group of cytokines containing members of the interferon family.

The peptides of the invention may be administered prophylactically in order to help to prevent the development of obesity, or any other leptin-related disorder.

The peptides in accordance with the invention may be administered in a dosage in the range 1 nM to 10 mM. A dosage unit of about 2 μM is typical. However, the optimum dosage will, of course, depend upon the condition being treated.

Each peptide of the combination of peptides in accordance with the present invention may be administered simultaneously or sequentially, in any therapeutically appropriate combination.

The combination of peptides may be formulated and administered as a single pharmaceutical composition comprising the peptides.

Alternatively each peptide may be formulated as separate compositions, which may then be administered simultaneously.

Alternatively the peptides of the combination may be administered sequentially. Preferably each peptide of the combination is administered within a biologically relevant time frame. In other words, one peptide of the combination is administered during the period when a patient is experiencing the biological effects of the administration of another peptide of the combination.

Each peptide of the combination is preferably present in equimolar ratios. However, it will be appreciated by the person skilled in the art that the ratios of the peptides in the combination may be varied. For instance, when two peptides are present in the combination, the molar ratios of one peptide to the other may vary from 1:1000 to 1000:1, more preferably 1:100 to 100:1, more preferably 1:10 to 10:1, more preferably 1:5 to 5:1, more preferably 1:4 to 4:1, more preferably 1:3 to 3:1, more preferably 1:2 to 2:1. The skilled person will appreciate other ratios may be desirable, depending on the therapeutic application. When there are 3 or more peptides present in the combination, they are preferably present in equimolar amounts. Alternatively, the relative amounts of each peptide may vary, depending on the therapeutic application.

The peptides in accordance with the invention may be formulated for administration in any suitable form. Thus, the use in accordance with the invention may be in the form of a composition, especially a pharmaceutical composition, which includes the peptide in combination with a physiologically acceptable carrier. The peptide may, for example, be formulated for oral, topical, rectal or parenteral administration. More specifically, the peptide may be formulated for administration by injection, or, preferably, in a form suitable for absorption through the mucosa of the oral/nasopharyngeal cavity, the alimentary canal or any other mucosal surface. The peptide may be formulated for administration intravenously, subcutaneously, or intramuscularly. The oral formulations may be provided in a form for swallowing or, preferably, in a form for dissolving in the saliva, whereby the formulation can be absorbed in the mucous membranes of the oral/nasopharyngeal cavity. The oral formulations may be in the form of a tablet (i.e. fast dissolving tablets) for oral administration, lozenges (i.e. a sweet-like tablet in a form suitable to be retained in the mouth and sucked), or adhesive gels for rubbing into the gum. The peptide may be formulated as an adhesive plaster or patch, which may be applied to the gums. The peptide may also be formulated for application to mucous-membranes of the genito-urinary organs. The topical formulations may be provided in the form of, for example, a cream or a gel. The peptide may also be formulated as a spray for application to the nasopharyngeal or bronchial mucous surface.

The peptides may be incorporated into products like milk, yogurts, milkshake, ice cream, cheese spread and various beverage products, including sport drinks.

In another aspect, the invention provides an antibody for the peptides, and provides compositions containing said antibodies. In particular the invention provides the antibodies in substantially isolated form. The antibodies can be produced by injecting a suitable subject, such as a rabbit, with the peptides (with a suitable adjuvant), then recovering the antibodies from the subject after allowing time for them to be produced. It is possible to test that the correct antibody has been produced by ELISA (enzyme-linked immunosorbent assay) using the synthetic peptide as antigens. The antibodies have potential uses in therapy, as a diagnostic tool and as a research tool. The antibodies can be produced in accordance with the methods described in example 3 of WO00/75173.

The invention also encompasses the selective administration of the peptides, at selected times to a patient.

In some applications it may be desirable to provide a pharmaceutical composition which contains the peptides in combination with a physiologically acceptable carrier.

The invention further embraces the use of the peptides in the manufacture of a medicament for use in any of the therapeutic applications described above.

The invention further embraces the methods of treating a mammal, in particular human, in any of the therapeutic applications described above.

### EXAMPLE 1

Mice, fed on a high-fat diet (HFD) for ten months, exhibited in a significant increase in body weight compared with mice kept on regular diet (54.7 g±4.6 g [HFD], compared to 29.8 g±2.3 g [regular diet], p<0.001).

Co-administration of peptides of SEQ ID NO 2, 9 and 10, alone or in any combination thereof, with HFD in mice for 10 months resulted in a lower body weight gain compared to control fed with HFD alone.

| Control [HFD, with none of peptides | 54.7 g | ± 4.6 g |
|---|---|---|
| SEQ ID NO 2, 9, or 10]: | | |
| Peptide of SEQ ID NO 2 with HFD: | 52.1 g | ± 2.3 g |
| Peptide of SEQ ID NO 9 with HFD: | 51.1 g | ± 2.3 g |
| Peptide of SEQ ID NO 10 with HFD: | 50.1 g | ± 2.3 g |
| Peptide of SEQ ID NO 2 and 9 with HFD: | 53.1 g | ± 1.1 g |
| Peptide of SEQ ID NO 2 and 10 with HFD: | 51.1 g | ± 1.7 g |
| Peptide of SEQ ID NO 9 and 10 with HFD: | 53.1 g | ± 1.1 g |
| Peptide of SEQ ID NO 2, 9 and 10 with HFD: | 46.1 | ± 1.3 g |

(p = 0.014)

Thus the peptides of SEQ ID NO 2, 9 and 10, alone or in combination, may have significance in the prevention and/or treatment of obesity, disorders associated with obesity, such as hyperphagy and type II diabetes mellitus, and leptin-associated disorders. The peptides may also be used to control body mass. It will be noted that mice administered with the combination of SEQ ID NO 2, 9 and 10 and fed with HFD exhibit a particularly low body weight gain.

It will be appreciated that the invention described herein may be modified, within the scope of the claims.

1-410. (canceled)

411. A medicament comprising:
(i) a peptide comprising the amino-terminal amino acid sequence RPKHPIKHQGLPQEVLNENLLRF (SEQ ID NO 1);
(ii) a salt of (i);
(iii) a peptide analogue of (i) in which one or more amino acids have been replaced, altered and/or deleted, provided the peptide analogue exhibits substantially the same biological properties as (i); or
(iv) a salt of (iii).

412. The medicament according to claim 411, wherein the medicament contains a peptide consisting of the amino acid sequence RPKHPIKHQGLPQEVLNENLLRF (SEQ ID NO 1) or a salt thereof.

413. The medicament according to claim 411, wherein the medicament is provided in a form suitable for oral, topical, rectal or parenteral administration, suitable for injection, or suitable for intravenous, subcutaneous, or intramuscular administration.

414. The medicament according to claim 411, wherein the peptide or the peptide analogue is obtained from a synthetic process.

415. The medicament according to claim 411, wherein the peptide or the peptide analogue is obtained from liquid-phase or solid-phase synthesis.

416. The medicament according to claim 411, wherein the peptide is isolated from a natural source.

417. The medicament according to claim 411, wherein the peptide or the peptide analogue is obtained by overexpression from a suitable plasmid containing a DNA sequence encoding the peptide or the peptide analogue transfected into a suitable host.

418. The medicament according to claim 411, wherein the medicament includes:
(1) a peptide which substantially consists of the amino acid sequence (X)ₙ-RPKHPIKHQGLPQEVLNENLLRF (SEQ ID NO 1)-(Z)ₘ, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence RPKHPIKHQGLPQEVLNENLLRF (SEQ ID NO 1), wherein n and m are the same or different, wherein m=0 or 1 and n=0 or 1;
(2) a salt of (1);
(3) a peptide analogue of (1) in which one or more amino acids have been replaced, altered and/or deleted without substantially altering the biological properties of (1); or
(4) a salt of (3).

419. The medicament according to claim 418 wherein X and/or Y contains ten or fewer amino acids.

420. The medicament according to claim 418 wherein X and/or Y contains five or fewer amino acids.

421. A composition comprising:
a first peptide or salt thereof selected from the group consisting of:
(1a) a peptide comprising the amino-terminal amino acid sequence RPKHPIKHQGLPQEVLNENLLRF (SEQ ID NO 1);
(1b) a salt of (1a);
(1c) a peptide analogue of (1a) in which one or more amino acids have been replaced, altered and/or deleted, provided the peptide analogue exhibits substantially the same biological properties as (1a); and
(1d) a salt of (1c); and
a second peptide or salt thereof that is different from the first peptide or salt thereof.

422. The composition according to claim 421, wherein the second peptide or salt thereof is selected from the group consisting of:
(2a) a peptide comprising the amino acid sequence RMPLPPRGCPAAAPWS (SEQ ID NO 10);
(2b) a salt of (2a);
(2c) a peptide analogue of (2a) in which one or more amino acids have been replaced, altered and/or deleted, provide the peptide analogue exhibits substantially the same biological properties as (2a); and
(2d) a salt of (2c).

423. The composition according to claim 421, wherein the second peptide or salt thereof is selected from the group consisting of:
(2e) a peptide which substantially consists of the amino acid sequence (X)ₙ-RMPLPPRGCPAAAPWS (SEQ ID NO 10)-(Z)ₘ, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence RMPLPPRGCPAAAPWS (SEQ ID NO 10), wherein n and m are the same or different, wherein m=0 or 1 and n=0 or 1;
(2f) a salt of (2e);
(2g) a peptide analogue of (2e) in which one or more amino acids have been replaced, altered and/or deleted without substantially altering the biological properties of (2e); and
(2h) a salt of (2g).

424. The composition according to claim 421, wherein the second peptide or salt thereof is selected from the group consisting of:
(3a) a peptide comprising the amino acid sequence SDIPNPIGSENSEKTTMPLW (SEQ ID NO 3);
(3b) a salt of (3a);
(3c) a peptide analogue of (3a) in which one or more amino acids have been replaced, altered and/or deleted, provide the peptide analogue exhibits substantially the same biological properties as (3a);
(3d) a salt of (3c);
(3e) a peptide which substantially consists of the amino acid sequence (X)ₙ-SDIPNPIGSENSEKTTMPLW (SEQ ID NO 3)-(Z)ₘ, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence SDIPNPIGSENSEKTTMPLW (SEQ ID NO 3), wherein n and m are the same or different, wherein m=0 or 1 and n=0 or 1;
(3f) a salt of (3e);
(3g) a peptide analogue of (3e) in which one or more amino acids have been replaced, altered and/or deleted without substantially altering the biological properties of (3e);
(3h) a salt of (3g);
(4a) a peptide comprising the amino acid sequence GPVRGPFPI (SEQ ID NO 4);
(4b) a salt of (4a);
(4c) a peptide analogue of (4a) in which one or more amino acids have been replaced, altered and/or deleted, provide the peptide analogue exhibits substantially the same biological properties as (4a);
(4d) a salt of (4c);
(4e) a peptide which substantially consists of the amino acid sequence (X)ₙ-GPVRGPFPI (SEQ ID NO 4)-(Z)ₘ, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence GPVRGPFPI (SEQ ID NO 4), wherein n and m are the same or different, wherein m=0 or 1 and n=0 or 1;
(4f) a salt of (4e);
(4g) a peptide analogue of (4e) in which one or more amino acids have been replaced, altered and/or deleted without substantially altering the biological properties of (4e);
(4h) a salt of (4g);
(5a) a peptide comprising the amino acid sequence EPVLGPVRGPFPI (SEQ ID NO 5);
(5b) a salt of (5a);
(5c) a peptide analogue of (5a) in which one or more amino acids have been replaced, altered and/or deleted, provide the peptide analogue exhibits substantially the same biological properties as (5a);
(5d) a salt of (5c);
(5e) a peptide which substantially consists of the amino acid sequence (X)ₙ-EPVLGPVRGPFPI (SEQ ID NO 5)-(Z)ₘ, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence EPVLGPVRGPFPI (SEQ ID NO 5), wherein n and m are the same or different, wherein m=0 or 1 and n=0 or 1;
(5f) a salt of (5e);
(5g) a peptide analogue of (5e) in which one or more amino acids have been replaced, altered and/or deleted without substantially altering the biological properties of (5e);
(5h) a salt of (5g);
(6a) a peptide comprising the amino acid sequence VPYPQRDMPIQ (SEQ ID NO 6);
(6b) a salt of (6a);
(6c) a peptide analogue of (6a) in which one or more amino acids have been replaced, altered and/or deleted, provide the peptide analogue exhibits substantially the same biological properties as (6a);
(6d) a salt of (6c);
(6e) a peptide which substantially consists of the amino acid sequence (X)ₙ-VPYPQRDMPIQ (SEQ ID NO 6)-(Z)ₘ, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence VPYPQRDMPIQ (SEQ ID NO 6), wherein n and m are the same or different, wherein m=0 or 1 and n=0 or 1;
(6f) a salt of (6e);
(6g) a peptide analogue of (6e) in which one or more amino acids have been replaced, altered and/or deleted without substantially altering the biological properties of (6e);
(6h) a salt of (6g);
(7a) a peptide comprising the amino acid sequence SLSQSKVLPVPQKAVPYPQRDMPIQ (SEQ ID NO 7);
(7b) a salt of (7a);
(7c) a peptide analogue of (7a) in which one or more amino acids have been replaced, altered and/or deleted, provide the peptide analogue exhibits substantially the same biological properties as (7a);
(7d) a salt of (7c);
(7e) a peptide which substantially consists of the amino acid sequence (X)ₙ-SLSQSKVLPVPQKAVPYPQRDMPIQ (SEQ ID NO 7)-(Z)ₘ, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence SLSQSKVLPVPQKAVPYPQRDMPIQ (SEQ ID NO 7), wherein n and m are the same or different, wherein m=0 or 1 and n=0 or 1;
(7f) a salt of (7e);
(7g) a peptide analogue of (7e) in which one or more amino acids have been replaced, altered and/or deleted without substantially altering the biological properties of (7e);
(7h) a salt of (7g);
(8a) a peptide comprising the amino acid sequence EPVLGPVR (SEQ ID NO 8);
(8b) a salt of (8a);
(8c) a peptide analogue of (8a) in which one or more amino acids have been replaced, altered and/or deleted, provide the peptide analogue exhibits substantially the same biological properties as (8a);
(8d) a salt of (8c);
(8e) a peptide which substantially consists of the amino acid sequence (X)ₙ-EPVLGPVR (SEQ ID NO 8)-(Z)ₘ, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence EPVLGPVR (SEQ ID NO 8), wherein n and m are the same or different, wherein m=0 or 1 and n=0 or 1;
(8f) a salt of (8e);
(8g) a peptide analogue of (8e) in which one or more amino acids have been replaced, altered and/or deleted without substantially altering the biological properties of (8e); and
(8h) a salt of (8g).

425. A method of treating a person afflicted with a disorder of the central nervous system, the method comprising:
administering a medicament to the person, wherein the medicament includes a therapeutically effective amount of
(i) a peptide comprising the amino-terminal amino acid sequence RPKHPIKHQGLPQEVLNENLLRF (SEQ ID NO 1);
(ii) a salt of (i);
(iii) a peptide analogue of (i) in which one or more amino acids have been replaced, altered and/or deleted, provided the peptide analogue exhibits substantially the same biological properties as (i); or
(iv) a salt of (iii).

426. The method according to claim 425, wherein the medicament contains a peptide consisting of the amino acid sequence RPKHPIKHQGLPQEVLNENLLRF (SEQ ID NO 1) or a salt thereof.

427. The method according to claim 425, wherein the medicament is provided in a form suitable for oral, topical, rectal or parenteral administration, suitable for injection, or suitable for intravenous, subcutaneous, or intramuscular administration.

428. The method according to claim 425, wherein the peptide is:
(1) a peptide which substantially consists of the amino acid sequence (X)ₙ-RPKHPIKHQGLPQEVLNENLLRF (SEQ ID NO 1)-(Z)ₘ, or a salt thereof, wherein X is an amino acid or peptide, Z is an amino acid or polypeptide, X and Z are the same or different, and wherein if (A) Z is glycine, X is not alanine, or (B) X is alanine, Z is not glycine, wherein X and Z do not substantially change the biological properties of a peptide consisting of an amino acid sequence RPKHPIKHQGLPQEVLNENLLRF (SEQ ID NO 1), wherein n and m are the same or different, wherein m=0 or 1 and n=0 or 1, or
(2) a peptide analogue of (1), or a salt thereof, in which one or more amino acids have been replaced, altered and/or deleted without substantially altering the biological properties of (1)

429. The method according to claim 428 wherein X and/or Y contains ten or fewer amino acids.

430. The method according to claim 428 wherein X and/or Y contains five or fewer amino acids.


SEQUENCE LISTING (10 sequences)

SEQ ID NO: 1 - protein, 23 aa, Ovis sp.
RPKHPIKHQGLPQEVLNENLLRF

SEQ ID NO: 2 - protein, 14 aa, Ovis sp.
FVAPFPEVFGKEKV

SEQ ID NO: 3 - protein, 20 aa, Ovis sp.
SDIPNPIGSENSEKTTMPLW

SEQ ID NO: 4 - protein, 9 aa, Ovis sp.
GPVRGPFPI

SEQ ID NO: 5 - protein, 13 aa, Ovis sp.
EPVLGPVRGPFPI

SEQ ID NO: 6 - protein, 11 aa, Ovis sp.
VPYPQRDMPIQ

SEQ ID NO: 7 - protein, 25 aa, Ovis sp.
SLSQSKVLPVPQKAVPYPQRDMPIQ

SEQ ID NO: 8 - protein, 8 aa, Ovis sp.
EPVLGPVR

SEQ ID NO: 9 - protein, 8 aa, Ovis sp.
RGPFPIIV

SEQ ID NO: 10 - protein, 16 aa, Ovis sp.
RMPLPPRGCPAAAPWS